(19) United States
(12) Patent Application Publication
(10) Pub. No.: US 2020/0009341 A1
(43) Pub. Date: Jan. 9, 2020

(54) APPARATUS AND METHOD FOR DIFFERENTIAL CAPACITIVE SENSING IN PATIENT'S TISSUE

(71) Applicant: HANSA MEDICAL PRODUCTS, INC., Carmel, IN (US)

(72) Inventors: Eric D. Blom, Carmel, IN (US); Zhen Huang, Carmel, IN (US); Larry Douglas O'Cull, Westfield, IN (US); Dart A. Fox, Indianapolis, IN (US); Brian Kamradt, Indianapolis, IN (US)

(21) Appl. No.: 16/553,146

(22) Filed: Aug. 27, 2019

Related U.S. Application Data
(63) Continuation-in-part of application No. 15/878,181, filed on Jan. 23, 2018.
Application No. 15/878,181 is a continuation of application No. 14/996,426, filed on Jan. 15, 2016, now abandoned.
Application No. 14/996,426 is a continuation-in-part of application No. 16/415,922, filed on May 17, 2019.
Application No. 16/415,922 is a division of application No. 15/452,323, filed on Mar. 7, 2017, now Pat. No. 10,293,129.
(60) Provisional application No. 62/103,842, filed on Jan. 15, 2015.
(60) Provisional application No. 62/115,097, filed on Feb. 11, 2015.
(60) Provisional application No. 62/118,674, filed on Feb. 20, 2015.
(60) Provisional application No. 62/364,812, filed on Jul. 20, 2016.
(60) Provisional application No. 62/304,756, filed on Mar. 7, 2016.

(51) Int. Cl. A61M 16/04; A61B 17/34; A61B 90/00; A61M 16/00; A61M 25/10

(52) U.S. Cl. CPC A61M 16/0472; A61B 17/3494; A61B 90/06; A61B 2017/00039; A61B 17/3415; A61M 25/10; A61M 16/0003

(57) ABSTRACT

A surgical instrument system for use in a surgical procedure is disclosed. The surgical instrument system may include an instrument configured to puncture the tissue of a patient and detect when the instrument has entered a lumen of the patient's body. The surgical instrument system may include one or more capacitive sensors near a tip of a needle and electrical circuitry to monitor the capacitance of the capacitive sensors. The capacitance of the capacitive sensors may change as the needle is inserted into different tissue of lumens of the patient's body, allowing for the position of the needle to be determined based on analysis of the capacitance of the capacitive sensors.

### CROSS-REFERENCES TO RELATED APPLICATIONS

This application claims priority under 35 U.S.C. § 120 to U.S. application Ser. No. 15/878,181, which was filed on Jan. 23, 2018, and claimed priority under 35 U.S.C. § 120 to U.S. application Ser. No. 14/996,426, which was filed on Jan. 15, 2016, and claimed priority under 35 U.S.C. § 119 to provisional application 62/103,842, which was filed on Jan. 15, 2015, provisional application 62/115,097, which was filed on Feb. 11, 2015, and provisional application 62/118,674, which was filed on Feb. 20, 2015.

This application claims priority under 35 U.S.C. § 120 to U.S. application Ser. No. 16/415,922, which was filed on May 17, 2019, and was a divisional of U.S. application Ser. No. 15/452,323, now U.S. Pat. No. 10,293,129, which was filed on Mar. 7, 2017, and claimed priority under 35 U.S.C. § 119 to provisional application 62/304,756, which was filed on Mar. 7, 2016, and provisional application 62/364,812, which was filed on Jul. 20, 2016. Each of the above-identified applications is expressly incorporated herein by reference.

### TECHNICAL FIELD

The present disclosure relates to instruments for capacitive sensing of a patient's tissue and, more specifically, in some embodiments, for detecting when a needle has entered a lumen of a patient.

### BACKGROUND

There are a number of techniques for establishing an adequate air passageway for a patient. When the trachea, nostrils and/or mouth are free of obstruction, endotracheal intubation, which involves the insertion of a tube through the nostrils or mouth and into the trachea itself, may be used. One endotracheal tube system for use in endotracheal intubation is described in International Patent Application Publication No. WO2014/088904, which is incorporated herein by reference.

Another technique for establishing an adequate air passageway involves the creation of a puncture or incision in the tracheal wall. A tracheostomy tube may then be inserted through the opening to form a passageway that effectively bypasses the upper trachea, nostrils and/or mouth. The initial incision may be made with a smaller needle and then enlarged or dilated to receive the tracheostomy tube.

Various techniques and devices for creating punctures or other incisions in the soft issue of a patient are illustrated and described in: “An Endoscopic Technique for Restoration of Voice After Laryngectomy,” Annals of Ontology, Rhinology And Laryngology, Singer et al., 89: 529-533, 1980, “Tracheoesophageal Puncture,” Atlas of Transnasal Esphagoscopy,” Postma, et al., 2007, Tracheoesophageal Puncture in the Office Setting with Local Anesthesia, Annals of Ontology, Rhinology And Laryngology, Desyatnikova et al., 110, 613-616, 2001, A Failsafe Technique For Tracheoesophagal Puncture, Koch, The Laryngoscope, 111, September 2001, A New Method For Tracheoesophagal Puncture Under Topical Anesthesia, Gross et al., The Laryngoscope, 104, February 1994, and the Blom-Singer® Voice Prosthesis Placement Surgical Kit available from Inhealth® Technologies. Another device for use with the soft tissue of a patient is the BD Angiocath Autoguard Shielded IV Catheter, which is commercially available from Becton, Dickinson and Company of New Jersey.

There are also the devices and methods illustrated and described in U.S. Pat. Nos. 5,653,230; 5,217,005; and 8,696,697; and U.S. Pat. App. Pub. No. 2012/0180787. The disclosures of these references are hereby incorporated herein by reference. This listing is not intended as a representation that a complete search of all relevant prior art has been conducted, or that no better references than those listed exist.

### SUMMARY

According to one aspect of the disclosure, a surgical instrument system for detecting a lumen in a patient's body is disclosed. The instrument system includes a surgical instrument that monitors changes in electrical capacitance normally present in tissue to determine when the instrument has entered the lumen. In one embodiment, the surgical instrument includes a needle extending from a handle. The entire instrument except for the cutting tip may be insulated. As the tip is advanced through tissue, the instrument detects fluctuating levels of electrical capacitance. When the tip enters, for example, an air-filled target lumen, a significant drop in electrical capacitance is detected, which the instrument associates with the entry of the tip into the lumen. When used to detect entry into other lumens of a patient's body, the device is configured to determine whether the fluctuating levels in electrical capacitance are greater than a predetermined threshold, which the instrument associates with the entry of the tip into a lumen.

When the instrument determines that the needle tip has entered the target lumen, the instrument may then activate an indicator such as, for example, a flashing light emitting diode (LED) in the instrument to alert the operator to not advance further. In one embodiment, the instrument may also be programmed to instantaneously retract its tip a distance of, for example, about 8 mm. In other embodiments, the tip of the instrument may remain stationary to facilitate fluid infusion or suction. In some embodiments, the system may include a noncompliant dilation balloon on a catheter for use in procedures such as, for example, percutaneous tracheostomy or percutaneous gastrostomy. In some embodiments, the surgical instrument may be another cutting tool such as, for example, a cutting blade in which the entire blade but a portion of the cutting edge may be insulated.

In some embodiments, the instrument is configured to differentiate between different types of tissue. For example, the instrument may be configured to differentiate between the electrical capacitance associated with an internal organ and the electrical capacitance associated with cancerous tissue. In such embodiments, the instrument may activate a visual or audible in the instrument to alert the operator that the tip has exited one type of tissue and entered a different type of tissue.

According to another aspect, a method for performing a surgical procedure comprises inserting a needle tip of a surgical instrument into a patient's tissue, advancing the needle tip through the tissue, monitoring an indicator of the surgical instrument while advancing the needle tip through the tissue, and maintaining a position of the surgical instrument in response to the indicator indicating the needle tip has entered a target lumen of the patient.

Illustratively according to this aspect, the surgical instrument may be operable to automatically retract the needle tip when the needle tip has entered the target lumen of the patient.

According to another aspect, a method for performing a surgical procedure comprises energizing an indicator of a surgical instrument to provide a first indication to a user when a needle tip is engaged with a portion of patient's tissue and energizing the indicator to provide a second indication different from the first indication in response to the needle tip exiting the portion of the patient's tissue.

Illustratively according to this aspect, the method may further comprise automatically retracting the needle tip in response to the needle tip exiting the portion of the patient's tissue.

Illustratively according to this aspect, energizing the indicator to provide the second indication different from the first indication in response to the needle tip exiting the portion of the patient's tissue includes energizing the indicator when the needle tip has entered the target lumen of the patient.

Illustratively according to this aspect, energizing the indicator to provide the second indication different from the first indication in response to the needle tip exiting the portion of the patient's tissue includes energizing the indicator when the needle tip has entered another portion of the patient.

According to another aspect, a method for performing a surgical procedure comprises inserting a needle tip of a surgical instrument into a first surface of a patient's tissue, advancing the needle tip through the tissue, monitoring an indicator of the surgical instrument while advancing the needle tip through the tissue, and withdrawing the needle tip from the tissue in response to the indicator indicating the needle tip has penetrated a second surface of the tissue.

Illustratively according to this aspect, the method comprises energizing a light source of the indicator prior to inserting the needle tip into the first surface. Further according to this aspect, inserting the needle tip of the surgical instrument into the first surface of the tissue comprises closing an electrical switch to de-energize the light source.

Illustratively according to this aspect, closing the electrical switch includes engaging an electrical contact with the tissue to couple a contact of the electrical switch with an electrically conductive component of the surgical instrument.

Illustratively according to this aspect, the method comprises advancing a body including an electrically conductive surface into an internal passageway of the patient, whereupon the needle tip contacts the electrically conductive component to energize a light source of the indicator.

Illustratively according to this aspect, advancing the needle tip through the tissue includes advancing the needle tip from a posterior wall of a patient's trachea through an anterior wall of the patient's esophagus.

Illustratively according to this aspect, the indicator indicates when the needle tip is positioned between the anterior wall of the patient's esophagus and a posterior wall of the patient's esophagus.

Illustratively according to this aspect, advancing the needle tip through the tissue includes advancing the needle tip through a patient's skin into a patient's trachea.

Illustratively according to this aspect, the indicator indicates when the needle tip is positioned between the anterior wall of the patient's trachea and a posterior wall of the patient's trachea

Illustratively according to this aspect, advancing the needle tip through the tissue includes advancing the needle tip from a first wall of a patient's tissue into a second wall of the patient's tissue.

Illustratively according to this aspect, the indicator indicates when the needle tip is positioned between the second wall of the patient's tissue and a third wall of the patient's tissue, the second wall and the third wall cooperating to define a lumen of the patient.

According to another aspect, a surgical instrument comprises an elongated body including a needle tip configured to puncture a patient's tissue, and an indicator secured to the elongated body, the indicator being operable to provide an indication of when the needle tip has penetrated an inner surface of the tissue.

Illustratively according to this aspect, the indicator includes a light source coupled to the elongated body.

Illustratively according to this aspect, the indicator includes an electrical switch moveable between an open position in which the light source is energized, and a closed position in which the light source is de-energized.

Illustratively according to this aspect, the elongated body includes a shaft comprising an electrically conductive material, and the electrical switch includes a conductive wire extending from the needle tip. The conductive wire includes a first end that is moveable between the open position in which the first end of the conductive wire is spaced apart from the electrically conductive material, and the closed position in which the first end of the conductive wire contacts the electrically conductive material.

Illustratively according to this aspect, the switch is biased in the open position.

Illustratively according to this aspect, the elongated body includes a handle and the shaft extends from the handle to the needle tip, and the indicator includes electrical circuitry in the handle.

Illustratively according to this aspect, the shaft is a cannula.

Illustratively according to this aspect, the indicator includes an electrical switch that is moveable between a closed position in which the light source is energized, and an open position in which the light source is de-energized.

Illustratively according to this aspect, a first electrical contact of the electrical switch includes the needle tip.

Illustratively according to this aspect, the surgical instrument further comprises a target body configured to be inserted into a passageway of the patient. The target body includes an electrically conductive surface. A second electrical contact of the electrical switch includes the electrically conductive surface such that when the needle tip engages the electrically conductive surface, the switch is closed.

Illustratively according to this aspect, the elongated body includes a handle and a shaft extending distally away from the handle to the needle tip, and the indicator includes electrical circuitry positioned in the handle.

According to another aspect, a surgical instrument comprises an elongated body including a handle and a shaft extending from the handle to a distal tip configured to puncture a patient's tissue, and an indicator including a light source in the handle. The light source is de-energized when the tip is located in the tissue between an external surface of the tissue and an internal surface of the tissue.

Illustratively according to this aspect, the shaft is electrically-conductive, and the indicator includes a conductor that is moveable between a position in which the conductor is spaced apart from the elongated body and the light source is energized, and a closed position in which the conductor engages the elongated body and the light source is de-energized.

According to another aspect, a surgical instrument comprises an elongated body including a handle and a shaft extending distally away from the handle to a needle tip configured to puncture a patient's tissue, and an indicator including a light source in the handle and a target body configured to be inserted into a passageway of the patient. The indicator is operable to energize the light source when the needle tip engages the target body.

According to another aspect, a surgical instrument comprises an elongated body including a handle and a shaft extending from the handle to a distal tip configured to puncture a patient's tissue, an indicator including a light source in the handle, and a capacitive sensor. The capacitive sensor is operable to energize the light source when the distal tip is located the tissue between an external surface of the tissue and an internal surface of the tissue.

According to another aspect, a surgical instrument comprises an elongated body including a handle and a shaft extending from the handle to a distal tip configured to puncture a patient's tissue, an indicator including a light source in the handle, and an electromagnetic sensor. The electromagnetic sensor is operable to energize the light source when the distal tip is located in the tissue between an external surface of the tissue and an internal surface of the tissue.

According to another aspect, a surgical instrument comprises an elongated body including a handle and a shaft extending from the handle to a distal tip configured to puncture a patient's tissue, an indicator including a light source in the handle, and a fiber optical thermometer needle sensor. The fiber optical thermometer needle sensor is operable to energize the light source when the distal tip is located in the tissue between an external surface of the tissue and an internal surface of the tissue.

According to another aspect, a surgical instrument comprises an elongated body including a handle and a shaft extending from the handle to a distal tip configured to puncture a patient's tissue, an indicator including a light source in the handle, a capacitive sensor operable to energize the light source when the distal tip penetrates a lumen of the patient's body, and a retraction mechanism operable to automatically retract the distal tip after the distal tip penetrates the lumen of the patient's body.

According to another aspect, a dilation instrument system is disclosed. The dilation instrument system includes a percutaneous dilation balloon and a moveably positionable retainer. The percutaneous dilation balloon is included in a balloon catheter configured to be positioned in an opening defined in a tracheal wall of a patient. The catheter includes a sheath having a proximal end and a distal end, the balloon extending over the sheath between the proximal end and the distal end, and a deflectable retention flange secured to the distal end of the sheath. The retainer is positioned over the balloon and is configured to move relative to the balloon such that upon inflation of the balloon when the balloon is positioned in the opening in the tracheal wall, the retainer engages the tracheal wall to inhibit movement of the balloon catheter.

According to another aspect, a dilation instrument system comprises a percutaneous dilation balloon, a stationary deflectable retention flange, and a moveably positionable retainer. The retainer is positioned over the balloon and is configured to move relative to the balloon such that upon inflation of the balloon when the balloon is positioned in an opening in a wall of the patient's tissue, the retainer is positioned adjacent to the wall to inhibit movement of the balloon catheter.

In some embodiments, the inflatable balloon may have a maximum diameter when inflated, and the retainer may include an annular body having an inner diameter that is less than the maximum diameter of the inflatable balloon. Additionally, in some embodiments, the annular body may include a first collar extending in a first direction, a second collar extending outwardly in a second direction opposite the first direction, and a passageway extending between an opening defined in the first collar and an opening defined in the second collar. The passageway may define the inner diameter of the annular body.

In some embodiments, the retainer may be formed from an elastomeric material. Additionally, in some embodiments, the elastomeric material may be a liquid silicone rubber. The elastomeric material may have a hardness in a range of 60 and 80 durometer (all hardnesses specified herein are Shore A unless otherwise specified).

In some embodiments, the balloon may be formed from a polymeric material. Additionally, in some embodiments, the polymeric material may be selected from a group consisting of polyethylene, polyurethane, and nylon.

In some embodiments, the sheath may comprise a tip positioned at the distal end and that is formed from a first material. The sheath may comprise an elongated body extending from the tip to the proximal end. The elongated body may be formed from a second material that is harder than the first material.

In some embodiments, the dilation instrument system may further comprise a surgical instrument configured to be coupled to the balloon catheter. The surgical instrument may comprise an elongated shaft sized to be positioned in a lumen defined in the sheath and a needle tip configured to puncture the tracheal wall. The needle tip may be configured to extend outwardly from the distal end of the sheath when the surgical instrument is coupled to the balloon catheter.

In some embodiments, the surgical instrument of the dilation instrument system may further comprise a handle coupled to the elongated shaft, an indicator including a light source in the handle, and a capacitive sensor operable to energize the light source when the needle tip penetrates a lumen of the patient's trachea.

In some embodiments, the surgical instrument of the dilation instrument system may comprise a retraction mechanism operable to automatically retract the needle tip after the needle tip penetrates the lumen of the patient's trachea.

According to another aspect, a surgical instrument system comprising a catheter having a lumen defined therein, the catheter further including a distal tip formed from a first material and an elongated body extending from the distal tip to an opposite proximal end, the elongated body being formed from a second material that has a hardness greater than the first material.

Additionally, in some embodiments, the first material may have a hardness in a range of 30 durometer to 50 durometer. In some embodiments, the elongated body may include a flared section at the proximal end.

According to another aspect, a method of dilating an opening in a patient's tissue is disclosed. The method comprises advancing a distal end of a balloon catheter in a first direction through the opening in the patient's tissue, pulling the balloon catheter in a second direction opposite the first direction to engage a retention flange secured to the distal end with an inner surface of the patient's tissue, advancing a retainer along the balloon catheter in the first direction to engage an outer surface of the patient's tissue opposite the inner surface, and inflating a balloon of the balloon catheter to dilate the opening in the patient's tissue.

According to another aspect, a method of dilating an opening in a patient's tissue comprises positioning an uninflated dilation balloon in the opening in the patient's tissue, engaging a retention flange with an inner surface of the patient's tissue, advancing a moveable retainer along the balloon to a position adjacent to an outer surface of the patient's tissue opposite the inner surface, and inflating the balloon to dilate the opening in the patient's tissue.

In some embodiments, the method may further comprise positioning an elongated shaft of a surgical instrument in a lumen defined in the balloon catheter such that a needle tip of the surgical instrument extends outwardly from the distal end of the balloon catheter, inserting the needle tip of the surgical instrument into the outer surface of the patient's tissue, and advancing the needle tip through the tissue to define the opening.

In some embodiments, the surgical instrument may be operable to automatically retract the needle tip into the lumen of the balloon catheter when the needle tip has penetrated the inner surface of the tissue.

In some embodiments, the method may further comprise monitoring an indicator of the surgical instrument while advancing the needle tip through the tissue. The surgical instrument may be operable to automatically retract the needle tip in response to the indicator indicating the needle tip has penetrated the inner surface of the tissue. Additionally, in some embodiments, the indicator may be operable to provide a visual indication when the needle tip has penetrated the inner surface of the tissue.

In some embodiments, advancing the retainer along the balloon catheter in the first direction may include engaging an annular body of the retainer with the outer surface of the tissue.

According to another aspect, a dilation instrument system is disclosed. The system comprises a balloon catheter configured to be positioned in an opening defined in a patient's tissue. The catheter includes a sheath having a proximal end and an elastomeric distal end, an inflatable balloon extending over the sheath between the proximal end and the distal end, and a deformable retention flange secured to the distal end of the sheath. The system also includes a retainer positioned over the balloon and configured to move relative to the balloon such that upon inflation of the balloon when the balloon is positioned in the opening in the patient's tissue, the retainer engages the patient's tissue to inhibit movement of the balloon catheter. The system also includes a surgical instrument removably coupled to the sheath. The surgical instrument comprises a needle tip extending outwardly from the sheath that is configured to puncture the patient's tissue. The surgical instrument may further comprise a retraction mechanism operable to automatically retract the needle tip after the needle tip penetrates a lumen of the patient's tissue. Additionally, the sheath may comprise a tip positioned at the distal end that is formed from a first material, and an elongated body extending from the tip to the proximal end of the sheath. The elongated body may be formed from a second material that is harder than the first material, and the retraction mechanism is operable to retract the needle tip into the tip of the sheath.

According to another aspect, a surgical instrument system comprises an elongated body including a handle and a shaft extending from the handle to a distal end configured to pass through a patient's tissue, an indicator including a light source, a sensor operable to generate an electrical signal, and a control circuit. The control circuit is configured to receive the electrical signal from the sensor, determine whether the distal end has penetrated a lumen of a patient, and energize the light source when the distal end has penetrated the lumen of the patient.

In some embodiments, the control circuit may be configured to determine whether the distal end has engaged tissue of a patient, energize the light source in a first state when the distal end has engaged tissue of the patient, and energize the light source in a second state when the distal end has penetrated the lumen of the patient. The second state may be different from the first state. Additionally, when the light source is in the first state, the light source may be flashing.

In some embodiments, the system may further comprise a retraction mechanism operable to retract the distal end. The control circuit may be configured to energize the retraction mechanism when the distal end has penetrated the lumen of the patient.

In some embodiments, the retraction mechanism may include a biasing element configured to bias the distal end in a retracted position. In some embodiments, the retraction mechanism may include a locking arm configured to maintain the distal end in an extended position.

The retraction mechanism may include an electric motor configured to move the locking arm to a disengaged position when the electric motor is energized. When the locking arm is in the disengaged position, the biasing element may be configured to urge the distal end to move to the retracted position.

According to another aspect, a surgical instrument system configured to perform any of the method described herein is disclosed.

According to one aspect, a surgical instrument system comprises a housing including a handle, a shaft extending outwardly from the housing to a distal end configured to form a puncture in a patient's tissue, a conductor plate positioned in the shaft, a retraction mechanism operable to move the distal end of the shaft in a first direction toward the housing, and a controller positioned in the housing. The controller is configured to energize a sensor circuit including a section of the shaft and the conductor plate, and monitor an electrical signal received from the sensor circuit. When an electrical resistance value based on the monitored electrical signal is greater than a predetermined threshold, the controller is configured to activate an indicator, and energize the retraction mechanism to move the distal end of the shaft in a direction toward the housing.

In some embodiments, the predetermined threshold for the resistance value may be greater than or equal to 100 kilo-ohms.

In some embodiments, the conductor plate may be positioned in an opening defined in the distal end of the shaft. Additionally, in some embodiments, the conductor plate may be a metallic inner shaft positioned in a passageway defined in the outer shaft.

In some embodiments, the system may comprise a non-conductive film positioned in the opening defined in the distal end of the shaft between the conductor plate and the shaft that electrically isolates the conductor plate from the shaft. Additionally, in some embodiments, the non-conductive film may include an annular ring that surrounds the conductor plate. In some embodiments, the non-conductive film may include a cylindrical ring that is positioned in a passageway defined in the outer shaft between a metallic inner shaft of the conductor plate and the outer shaft. In some embodiments, the annular ring or cylindrical ring may have a thickness of 0.5 millimeters. The non-conductive film may be formed from a non-conductive plastic or silicone material.

In some embodiments, the indicator is a visual indicator. Additionally, in some embodiments, the controller may be configured to determine whether the distal end has engaged tissue of a patient based on the electrical signal received from the sensor circuit, energize the indicator in a first state when the controller has determined that the distal end has engaged tissue of the patient, and energize the indicator in a second state to activate the indicator when the controller has determined that the distal end has penetrated the lumen of the patient. In some embodiments, the second state is different from the first state such that a user may determine whether the instrument is armed and/or has penetrated the lumen.

In some embodiments, when the resistance value based on the monitored electrical signal is less than a predetermined value for a predetermined period of time, the controller may be configured to energize the indicator in a first state to indicate the instrument is armed. Additionally, in some embodiments, the predetermined value may be in a range of 1 kilo-ohm to 100 kilo-ohms. In some embodiments, the predetermined period of time may be equal to 200 milliseconds. Additionally, in some embodiments, the instrument may include a switch operable to be toggled by a user to disarm the instrument.

In some embodiments, the first state may be one of a flashing light and a continuous light, and the second state may be the other of a flashing light and a continuous light. It should be appreciated that in some embodiments the first state may include flashing the indicator at a first frequency, and the second state may include flashing the indicator at a second frequency different from the first frequency.

In some embodiments, the retraction mechanism may include a linear actuator that is electrically-operated. Additionally, in some embodiments, the shaft may be operable to move along a first axis, and the linear actuator may be operable to move along a second axis extending orthogonal to the first axis to cause the shaft to move along the first axis.

In some embodiments, the shaft may extend from the distal end to a proximal end positioned in the housing, and the retraction mechanism includes a mounting frame secured to the proximal end of the shaft.

Additionally, in some embodiments, the retraction mechanism may include a locking arm operable to rotate about a pivot pin between a first position in which a proximal end of the mounting frame is engaged with a first surface of the locking arm and a second position in which the proximal end of the mounting frame is received in a passageway defined in the shaft. In some embodiments, the linear actuator is operable to advance into contact with the locking arm to cause the locking arm to rotate between the first position and the second position.

In some embodiments, the retraction mechanism may further comprise a biasing element attached to an end of the locking arm, and the biasing element may be operable to bias the locking arm in the first position.

In some embodiments, the mounting frame may include a mounting bracket that has a first end secured to the shaft and a second, opposite end secured to an elongated rod, and the elongated rod may include the proximal end of the mounting frame.

In some embodiments, the locking arm may include a sleeve that includes the first surface. Additionally, in some embodiments, retraction mechanism may further comprise a biasing element operable to urge the shaft in the first direction, and the biasing element may be positioned between a plate of the mounting frame and a wall of the housing.

According to another aspect, a method for performing a surgical procedure is disclosed. The method includes inserting a needle tip of a surgical instrument into a patient's tissue, advancing the needle tip through the tissue, monitoring an indicator of the surgical instrument while advancing the needle tip through the tissue, and maintaining a position of the surgical instrument in response to the indicator indicating the needle tip has entered a target lumen of the patient.

In some embodiments, the surgical instrument may be operable to automatically retract the needle tip when the needle tip has entered the target lumen of the patient. Additionally, in some embodiments, the surgical instrument may include a control circuit operable to measure a change in electrical resistance to determine when the needle tip has entered the target lumen of the patient and activate the indicator to indicate the needle tip has entered a target lumen of the patient.

According to another aspect, a method of performing a surgical procedure comprises energizing a sensor circuit of a surgical instrument including a needle tip configured for insertion into a patient's tissue, monitoring an electrical signal received from the sensor circuit, energizing an indicator in a first state when a resistance value based on the electrical signal is less than a predetermined value corresponding to the needle tip being positioned in the patient's tissue, energizing the indicator in a second state when the resistance value based on the electrical signal is greater than a predetermined threshold corresponding to the needle tip being positioned in a patient's lumen, and energizing a retraction mechanism of the surgical instrument to move the needle tip away from the patient's lumen.

In some embodiments, the method may further comprising activating a timer when the resistance value based on the electrical signal is less than a predetermined value. The step of energizing the indicator in the first state may include energizing the indicator in the first state after a predetermined amount of time has elapsed from the activation of the timer.

In some embodiments, the method may further include activating a timer when the resistance value based on the electrical signal is greater than a predetermined threshold. The step of energizing the retraction mechanism of the surgical instrument may include energizing the retraction mechanism of the surgical instrument after a predetermined amount of time has elapsed from the activation of the timer.

Additionally, in some embodiments, the surgical instrument may include an elongated shaft, and the sensor circuit may include a portion of the shaft and a conductor plate or shaft positioned in the shaft. In some embodiments, the sensor circuit may include a pair of conductor plates, and the elongated shaft may be formed from a non-conductive material.

According to another aspect of the disclosure, a surgical instrument system for detecting a lumen in a patient's body is disclosed. When the instrument determines that the needle tip has entered the target lumen, the instrument may then activate an indicator such as, for example, a flashing light emitting diode (LED) in the instrument to alert the operator to not advance further. In one embodiment, the instrument may also be programmed to instantaneously retract its tip a distance of, for example, about 8 mm In other embodiments, the tip of the instrument may remain stationary to facilitate fluid infusion or suction. In some embodiments, the system may include a noncompliant dilation balloon on a catheter for use in procedures such as, for example, percutaneous tracheostomy or percutaneous gastrostomy. In some embodiments, the surgical instrument may be another cutting tool such as, for example, a cutting blade in which the entire blade but a portion of the cutting edge may be insulated.

According to another aspect, a method for performing a surgical procedure comprises inserting a needle tip of a surgical instrument into a patient's tissue, advancing the needle tip through the tissue, monitoring an indicator of the surgical instrument while advancing the needle tip through the tissue, and maintaining a position of the surgical instrument in response to the indicator indicating the needle tip has entered a target lumen of the patient.

Illustratively according to this aspect, the surgical instrument may be operable to automatically retract the needle tip when the needle tip has entered the target lumen of the patient.

According to another aspect, a method for performing a surgical procedure comprises energizing an indicator of a surgical instrument to provide a first indication to a user when a needle tip is engaged with a portion of patient's tissue and energizing the indicator to provide a second indication different from the first indication in response to the needle tip exiting the portion of the patient's tissue.

Illustratively according to this aspect, the method may further comprise automatically retracting the needle tip in response to the needle tip exiting the portion of the patient's tissue.

Illustratively according to this aspect, energizing the indicator to provide the second indication different from the first indication in response to the needle tip exiting the portion of the patient's tissue includes energizing the indicator when the needle tip has entered the target lumen of the patient.

Illustratively according to this aspect, energizing the indicator to provide the second indication different from the first indication in response to the needle tip exiting the portion of the patient's tissue includes energizing the indicator when the needle tip has entered another portion of the patient.

According to another aspect, a dilation instrument system is disclosed. The dilation instrument system includes a percutaneous dilation balloon and a moveably positionable retainer. The percutaneous dilation balloon is included in a balloon catheter configured to be positioned in an opening defined in a tracheal wall of a patient. The catheter includes a sheath having a proximal end and a distal end, the balloon extending over the sheath between the proximal end and the distal end, and a deflectable retention flange secured to the distal end of the sheath. The retainer is positioned over the balloon and is configured to move relative to the balloon such that upon inflation of the balloon when the balloon is positioned in the opening in the tracheal wall, the retainer engages the tracheal wall to inhibit movement of the balloon catheter.

According to another aspect, a dilation instrument system comprises a percutaneous dilation balloon, a stationary deflectable retention flange, and a moveably positionable retainer. The retainer is positioned over the balloon and is configured to move relative to the balloon such that upon inflation of the balloon when the balloon is positioned in an opening in a wall of the patient's tissue, the retainer is positioned adjacent to the wall to inhibit movement of the balloon catheter.

In some embodiments, the inflatable balloon may have a maximum diameter when inflated, and the retainer may include an annular body having an inner diameter that is less than the maximum diameter of the inflatable balloon. Additionally, in some embodiments, the annular body may include a first collar extending in a first direction, a second collar extending outwardly in a second direction opposite the first direction, and a passageway extending between an opening defined in the first collar and an opening defined in the second collar. The passageway may define the inner diameter of the annular body.

In some embodiments, the sheath may comprise a tip positioned at the distal end and that is formed from a first material. The sheath may comprise an elongated body extending from the tip to the proximal end. The elongated body may be formed from a second material that is harder than the first material.

In some embodiments, the dilation instrument system may further comprise a surgical instrument configured to be coupled to the balloon catheter. The surgical instrument may comprise an elongated shaft sized to be positioned in a lumen defined in the sheath and a needle tip configured to puncture the tracheal wall. The needle tip may be configured to extend outwardly from the distal end of the sheath when the surgical instrument is coupled to the balloon catheter.

In some embodiments, the surgical instrument of the dilation instrument system may further comprise a handle coupled to the elongated shaft, an indicator including a light source in the handle, and a sensor operable to energize the light source when the needle tip penetrates a lumen of the patient's trachea.

In some embodiments, the surgical instrument of the dilation instrument system may comprise a retraction mechanism operable to automatically retract the needle tip after the needle tip penetrates the lumen of the patient's trachea.

According to another aspect, a surgical instrument system comprising a catheter having a lumen defined therein, the catheter further including a distal tip formed from a first material and an elongated body extending from the distal tip to an opposite proximal end, the elongated body being formed from a second material that has a hardness greater than the first material.

According to another aspect, a method of dilating an opening in a patient's tissue is disclosed. The method comprises advancing a distal end of a balloon catheter in a first direction through the opening in the patient's tissue, pulling the balloon catheter in a second direction opposite the first direction to engage a retention flange secured to the distal end with an inner surface of the patient's tissue, advancing a retainer along the balloon catheter in the first direction to engage an outer surface of the patient's tissue opposite the inner surface, and inflating a balloon of the balloon catheter to dilate the opening in the patient's tissue.

According to another aspect, a method of dilating an opening in a patient's tissue comprises positioning an uninflated dilation balloon in the opening in the patient's tissue, engaging a retention flange with an inner surface of the patient's tissue, advancing a moveable retainer along the balloon to a position adjacent to an outer surface of the patient's tissue opposite the inner surface, and inflating the balloon to dilate the opening in the patient's tissue.

In some embodiments, the method may further comprise positioning an elongated shaft of a surgical instrument in a lumen defined in the balloon catheter such that a needle tip of the surgical instrument extends outwardly from the distal end of the balloon catheter, inserting the needle tip of the surgical instrument into the outer surface of the patient's tissue, and advancing the needle tip through the tissue to define the opening.

In some embodiments, the surgical instrument may be operable to automatically retract the needle tip into the lumen of the balloon catheter when the needle tip has penetrated the inner surface of the tissue.

In some embodiments, the method may further comprise monitoring an indicator of the surgical instrument while advancing the needle tip through the tissue. The surgical instrument may be operable to automatically retract the needle tip in response to the indicator indicating the needle tip has penetrated the inner surface of the tissue. Additionally, in some embodiments, the indicator may be operable to provide a visual indication when the needle tip has penetrated the inner surface of the tissue.

In some embodiments, advancing the retainer along the balloon catheter in the first direction may include engaging an annular body of the retainer with the outer surface of the tissue.

According to another aspect, a dilation instrument system is disclosed. The system comprises a balloon catheter configured to be positioned in an opening defined in a patient's tissue. The catheter includes a sheath having a proximal end and an elastomeric distal end, an inflatable balloon extending over the sheath between the proximal end and the distal end, and a deformable retention flange secured to the distal end of the sheath. The system also includes a retainer positioned over the balloon and configured to move relative to the balloon such that upon inflation of the balloon when the balloon is positioned in the opening in the patient's tissue, the retainer engages the patient's tissue to inhibit movement of the balloon catheter. The system also includes a surgical instrument removably coupled to the sheath. The surgical instrument comprises a needle tip extending outwardly from the sheath that is configured to puncture the patient's tissue. The surgical instrument may further comprise a retraction mechanism operable to automatically retract the needle tip after the needle tip penetrates a lumen of the patient's tissue. Additionally, the sheath may comprise a tip positioned at the distal end that is formed from a first material, and an elongated body extending from the tip to the proximal end of the sheath. The elongated body may be formed from a second material that is harder than the first material, and the retraction mechanism is operable to retract the

According to another aspect, a surgical instrument system comprises an elongated body including a handle and a shaft extending from the handle to a distal end configured to pass through a patient's tissue, an indicator, a sensor operable to generate an electrical signal, and a control circuit. The control circuit is configured to receive the electrical signal from the sensor, determine whether the distal end has penetrated a lumen of a patient, and activate the indicator when the distal end has penetrated the lumen of the patient.

In some embodiments, the control circuit may be configured to determine whether the distal end has engaged tissue of a patient, energize the light source in a first state when the distal end has engaged tissue of the patient, and energize the light source in a second state when the distal end has penetrated the lumen of the patient. The second state may be different from the first state. Additionally, when the light source is in the first state, the light source may be flashing.

In some embodiments, the system may further comprise a retraction mechanism operable to retract the distal end. The control circuit may be configured to energize the retraction mechanism when the distal end has penetrated the lumen of the patient.

In some embodiments, the retraction mechanism may include a biasing element configured to bias the distal end in a retracted position. In some embodiments, the retraction mechanism may include a locking arm configured to maintain the distal end in an extended position.

In some embodiments, the control circuit may be configured to determine whether the distal end has engaged tissue of a patient, energize the light source in a first state when the distal end has engaged tissue of the patient, and energize the light source in a second state when the distal end has penetrated the lumen of the patient, the second state being different from the first state. In some embodiments, the system may further comprise a retraction mechanism operable to retract the distal end. The control circuit may be configured to energize the retraction mechanism when the distal end has penetrated the lumen of the patient.

In some embodiments, the sensor may include an outer surface of the shaft electrically connected to the control circuit and a first plate positioned at the distal end of the shaft. The first plate may be electrically connected to the control circuit.

Additionally, in some embodiments, the control circuit may be operable to apply an electrical charge to the first plate. In some embodiments, the sensor may be operable to measure changes in electrical properties of the patient's tissue.

In some embodiments, the sensor may be operable to measure changes in resistance.

According to another aspect, a method for performing a surgical procedure comprises inserting a needle tip of a surgical instrument into a patient's tissue, advancing the needle tip through the tissue, monitoring an indicator of the surgical instrument while advancing the needle tip through the tissue, and maintaining a position of the surgical instrument in response to the indicator indicating the needle tip has entered a target lumen of the patient.

In some embodiments, the surgical instrument may be operable to automatically retract the needle tip when the needle tip has entered the target lumen of the patient. Additionally, in some embodiments, the surgical instrument may include a control circuit operable to measure a change in electrical properties to determine when the needle tip has entered the target lumen of the patient and activate the indicator to indicate the needle tip has entered a target lumen of the patient.

In some embodiments, the surgical instrument may be operable to apply an electrical charge to a plate positioned at the needle tip. In some embodiments, the control circuit may be operable to determine when the needle tip has entered the target lumen of the patient based on a change in electrical resistance.

In some embodiments, the control circuit may be operable to determine when the needle tip has entered the target lumen of the patient based on a change in resistance. In some embodiments, the target lumen may be devoid of liquid and/or tissue. In some embodiments, liquid may be present in the target lumen.

According to another aspect, a surgical instrument system comprises a housing including a handle, a shaft extending outwardly from the housing to a distal end configured to form a puncture in a patient's tissue, the shaft comprising a capacitive sensor, and a controller positioned in the housing, the controller configured to measure a capacitance value of the capacitive sensor.

In some embodiments, the surgical instrument system further comprises a retraction mechanism operable to move the distal end of the shaft in a first direction toward the housing, wherein the controller is configured to (i) determine, based on the capacitance value of the capacitive sensor, whether the shaft should be retracted, and (ii) energize the retraction mechanism to move to the distal end of the shaft in the direction toward the housing in response to a determination that the shaft should be retracted.

In some embodiments, the surgical instrument system further comprises a light source or an audio source positioned in the housing, wherein the controller is configured to (i) determine, based on the capacitance value of the capacitive sensor, whether an alert should be provided, and (ii) energize the light source or the audio source to provide the alert in response to a determination that the alert should be provided.

In some embodiments, the shaft further comprises a second capacitive sensor electrically isolated from the capacitive sensor, wherein the controller is configured to measure a capacitance value of the second capacitive sensor.

In some embodiments, the surgical instrument system has a central tube positioned within the shaft.

In some embodiments, electrical wiring positioned within the central tube provides an electrical connection between the controller and the capacitive sensor and an electrical connection between the controller and the second capacitive sensor.

In some embodiments, the central tube is configured to act as an electrical ground-guard-shield for the capacitive sensor the second capacitive sensor.

In some embodiments, the controller is configured to determine when the distal end of the shaft has engaged a first tissue layer of a patient in response to the difference between the monitored capacitance values of the capacitive sensor and the second capacitive sensor being greater than a first predetermined threshold, and determine when the distal end has engaged a second tissue layer of a patient different from the first in response to the difference between the monitored capacitance values of the capacitive sensor and the second capacitive sensor being greater than a second predetermined threshold.

In some embodiments, the shaft further comprises a third capacitive sensor electrically isolated from the capacitive sensor and the second capacitive sensor, wherein the third capacitive sensor has a base capacitance value at least twice a base capacitance value of the capacitive sensor.

According to another aspect, a surgical instrument comprises a hollow shaft having a proximal end configured to couple to a housing and a distal end configured to form a puncture in a patient's tissue, wherein the hollow shaft comprises a capacitive sensor. The surgical instrument may further comprise an electrical connector electrically coupled to the capacitive sensor and extending along the hollow shaft from the capacitive sensor towards the proximal end of the hollow shaft.

In some embodiments, the hollow shaft comprises a second capacitive sensor, wherein the second capacitive sensor is electrically isolated from the capacitive sensor, and the surgical instrument further comprising a second electrical connector electrically coupled to the second capacitive sensor and extending along the hollow shaft from the second capacitive sensor towards the proximal end of the hollow shaft.

In some embodiments, each of the capacitive sensor and the second capacitive sensor are positioned within 25 millimeters of the distal end of the hollow shaft.

In some embodiments, each of the capacitive sensor and the second capacitive sensor are positioned within 5 millimeters of the distal end of the hollow shaft.

In some embodiments, the hollow shaft further comprises a third capacitive sensor electrically isolated from the capacitive sensor and the second capacitive sensor, wherein the third capacitive sensor has a base capacitance value at least twice a base capacitance value of the capacitive sensor.

In some embodiments, a central tube is positioned within the hollow shaft.

In some embodiments, the surgical instrument further comprises the housing, wherein the proximal end of the hollow shaft is coupled to the housing, and a controller positioned in the housing that is configured to measure a capacitance value of the capacitive sensor and a capacitance value of the second capacitive sensor.

According to another aspect, a method of using a surgical instrument comprises monitoring, by electrical circuitry of the surgical instrument, a capacitance of one or more capacitive sensors disposed on a shaft of the surgical instrument; analyzing, by the electrical circuitry, the capacitance of the one or more capacitive sensors; determining, by the electrical circuitry and based on the analysis of the capacitance of the one or more capacitive sensors, that the shaft of the surgical instrument should be retracted; and controlling, by the electrical circuitry, an actuator of the surgical instrument to retract the shaft.

In some embodiments wherein monitoring the capacitance of the one or more capacitive sensors comprises monitoring the capacitance of a first capacitive sensor and a second capacitive sensor, and analyzing the capacitance of the one or more capacitive sensors comprises comparing the capacitance of the first capacitive sensor to the capacitance of the second capacitive sensor.

In some embodiments, the method further comprises determining, by the electrical circuitry and based on the capacitance value of the one or more capacitive sensors, whether an alert should be provided; and energizing, by the electrical circuitry a light source or audio source of the surgical instrument to provide the alert in response to a determination that the alert should be provided.

In some embodiments, each of the one or more capacitive sensors are positioned within 25 millimeters of a distal end of the shaft opposite a proximal end of the shaft that is coupled to a handle of the surgical instrument.

According to another aspect, a surgical instrument system configured to perform any of the methods described herein is disclosed.

### BRIEF DESCRIPTION OF THE DRAWINGS

The detailed description particularly refers to the following figures, in which:

FIGS. 1A and B are a perspective view of one embodiment of a surgical instrument system for use in performing a surgical procedure and an enlarged perspective view of a detail of FIG. 1A;

FIG. 2 illustrates a circuit diagram of an electrical circuit of the surgical instrument system of FIG. 1;

FIG. 3 illustrates a sectional side elevation view of the surgical instrument system of FIG. 1 forming a puncture in tissue of a patient;

FIG. 4 illustrates a view similar to FIG. 3 showing a distal end of the surgical instrument system having reached a passageway beyond the tissue;

FIG. 5 illustrates a perspective view of another embodiment of a surgical instrument system;

FIG. 6 illustrates a circuit diagram of an electrical circuit of the surgical instrument system of FIG. 5;

FIG. 7 illustrates a cross sectional side elevation view of the surgical instrument system of FIG. 5 forming a puncture in the soft tissue of a patient;

FIG. 8 illustrates a view similar to FIG. 7 showing a distal end of the surgical instrument system having reached a passageway beyond the tissue;

FIG. 9A is a perspective view of another embodiment of a surgical instrument system for use in performing a surgical procedure;

FIG. 9B is an enlarged cross sectional view of a detail of the surgical instrument system of FIG. 9A;

FIG. 10 illustrates a circuit diagram of an electrical circuit of the surgical instrument system of FIG. 9;

FIGS. 11A and B are a perspective view of another embodiment of a surgical instrument system for use in performing a surgical procedure and an enlarged perspective view of a detail of FIG. 11A;

FIG. 12A is a perspective view of another embodiment of a surgical instrument system for use in performing a surgical procedure;

FIG. 12B is an enlarged cross sectional view of a detail of the surgical instrument system of FIG. 9A;

FIG. 13 illustrates a circuit diagram of an electrical circuit of the surgical instrument system of FIG. 12;

FIG. 14 is a perspective view illustrating a surgical instrument system for forming and dilating an opening in a patient's tissue;

FIG. 15 is a perspective view illustrating a balloon catheter and a retainer of the instrument system of FIG. 14;

FIG. 16 is a view similar to FIG. 15 illustrating the balloon catheter with the balloon inflated;

FIG. 17 is a side elevation view of a sheath of the balloon catheter of FIG. 15;

FIG. 18 is a cross-sectional elevation view of the balloon catheter taken along the line 18-18 in FIG. 16 with the retainer removed;

FIG. 19 is a partial cross-sectional plan view of a surgical instrument of the instrument system of FIG. 14;

FIG. 20 is an enlarged cross sectional view of a detail of FIG. 14;

FIG. 21 illustrates the needle retraction mechanism of the surgical instrument of FIG. 14;

FIG. 22 illustrates the needle retraction mechanism of FIG. 21 in an extended position;

FIG. 23 illustrates a circuit diagram of an electrical circuit of the surgical instrument system of FIG. 14;

FIG. 24 illustrates a perspective view of another embodiment of a surgical instrument system for use in performing a surgical procedure;

FIG. 25 illustrates a perspective view of some of the components of the system of FIG. 24; and

FIG. 26 illustrates a circuit diagram of an electrical circuit of the surgical instrument system of FIG. 24.

FIG. 27 is a perspective view illustrating another surgical instrument system;

FIG. 27A is a partial cross-section elevation view of a detail of FIG. 27;

FIGS. 28-29 are partial cross-sectional plan views of a surgical instrument of the instrument system of FIG. 27; and

FIG. 30 is a circuit diagram of an electrical circuit of the surgical instrument system of FIG. 27;

FIG. 31 is a side elevation view of the surgical instrument of FIGS. 27-30 positioned for insertion into a patient's soft tissue;

FIG. 32 illustrates the surgical instrument of FIGS. 27-30 as it enters a lumen of the patient;

FIG. 33 illustrates the surgical instrument of FIGS. 27-30 after the needle of the surgical instrument has been retracted;

FIG. 34 is a circuit diagram of an electrical circuit for the surgical instrument system of FIG. 27;

FIGS. 35A and B are a perspective view of one embodiment of a surgical instrument system for use in performing a surgical procedure and an enlarged perspective view of one embodiment of a detail of FIG. 35A;

FIGS. 36A and B are an enlarged longitudinal cross sectional view of the one embodiment of the detail of FIG. 35A and an enlarged transverse cross sectional view of the one embodiment of the detail of FIG. 35A;

FIGS. 37A and B are an enlarged perspective view of another embodiment of a detail of FIG. 35A and an enlarged longitudinal cross sectional view of the other embodiment of the detail of FIG. 35A;

FIG. 38 is a circuit diagram of an electrical circuit for the surgical instrument system of FIG. 35A;

FIGS. 39-40 are a flow chart of one embodiment of a method that may be performed by the surgical instrument system of FIG. 35A; and

FIG. 41 is a flow chart of one embodiment of a method that may be performed by the surgical instrument system of FIG. 35A.

### DETAILED DESCRIPTION OF THE DRAWINGS

While the concepts of the present disclosure are susceptible to various modifications and alternative forms, specific exemplary embodiments thereof have been illustrated by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

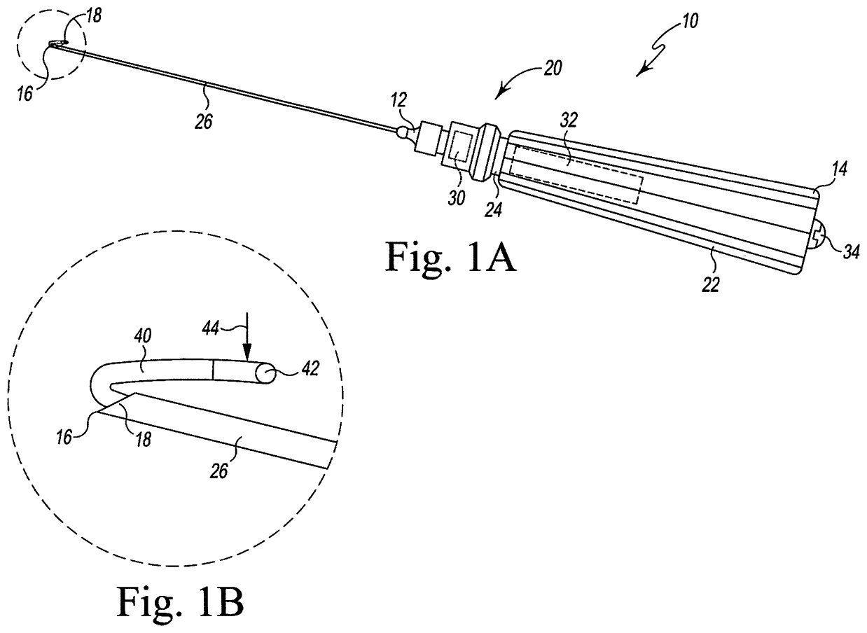

Referring now to FIGS. 1A-B, a surgical instrument system for detecting a lumen in a patient's body is illustrated. The system 10 includes a surgical instrument 10 that is configured for insertion into the soft tissue of a patient. Illustratively, the surgical instrument 10 may be use to form a puncture between the skin of the neck and the anterior wall of the trachea of a patient, but it should be appreciated that the surgical instrument 10 may be used to form other punctures, incisions, or openings in the patient's tissue. As illustrated in FIGS. 1A-B, the surgical instrument 10 includes an elongated body 12 having a proximal end 14 and a distal end 16. A needle tip 18 configured to pierce the tissue is formed at the distal end 16 of the body 12. As described in greater detail below, the surgical instrument 10 also includes an indicator 20 configured to notify a user that the needle tip 18 has penetrated the tissue.

The elongated body 12 includes a handle 22 extending from the proximal end 14 to a distal handle end 24. A shaft 26 extends distally away from the handle 22 to the needle tip 18. In the illustrated embodiment, the shaft 26 is a cannula formed from an electrically conductive material. The needle tip 18 and the shaft 26 are integral, but it should be appreciated that in other embodiments the needle tip 18 and the shaft 26 may be formed as separate components and assembled.

As illustrated in FIG. 1A, the indicator 20 is positioned at the distal handle end 24. In the illustrated embodiment, the indicator 20 includes a light source such as, for example, a light emitting diode (LED) 30, and other electrical circuitry 32 operable to energize the LED 30 to provide a visual output to the user. In other embodiments, the indicator 20 may include other electrical circuitry to provide an audible output to the user. The surgical instrument 10 also includes a power switch 34 positioned at the proximal end 14 of the handle 22. Power switch 34 is operable to supply power to the electrical circuitry 32 including LED 30.

The electrical circuitry 32 also includes a conductor 40 that extends down the shaft 26 and outwardly from the needle tip 18 to an end 42 spaced apart from the shaft 26. When sufficient force is applied in the direction indicated by arrow 44, the end 42 of the conductor 40 is pressed into contact with the shaft 26. The conductor 40 is elastic, so that when the force is removed, the conductor 40 returns to a position with the end 42 out of electrical contact with the shaft 26. In the illustrative embodiment, the conductor 40 is formed from conductive metallic spring wire.

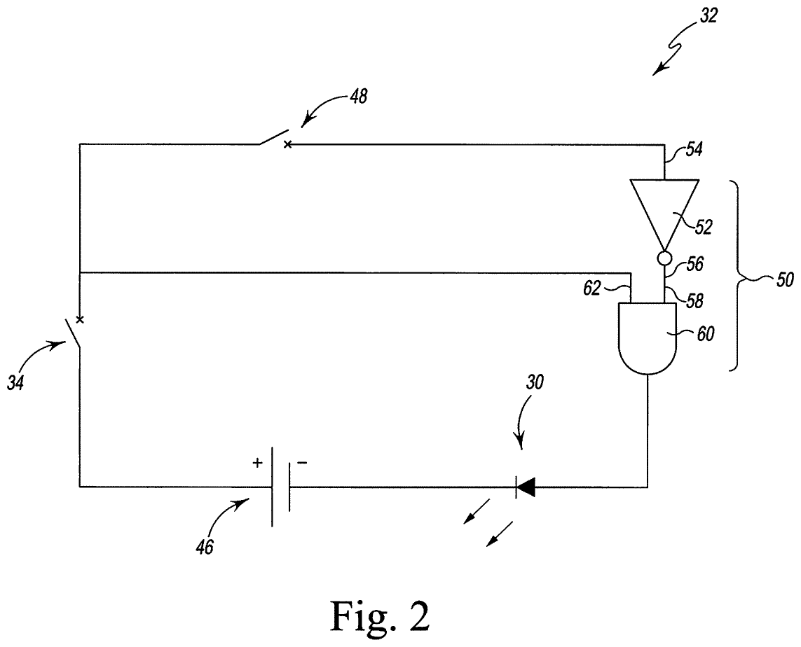

Referring now to FIG. 2, the electrical circuitry 32 includes a battery 46 configured to supply electrical power when switch 34 is closed. In the illustrative embodiment, the conductor 40 and the shaft 26 comprise the electrical contacts of an electrical switch 48 between switch 34 and a logic circuit 50 operable to control the supply of electrical power to the LED 30. The illustrative logic circuit 50 comprises an inverting amplifier and an AND gate, but the logic circuit 50 may be realized as other circuitry as appropriate.

The logic components 50 include inverting amplifier 52 configured to output an inverted version of its input. As illustrated in FIG. 2, the electrical switch 48 (i.e., the conductor 40 and the shaft 26) is coupled to the input 54 of the inverting amplifier 52. When the end 42 of the conductor 40 is spaced apart from the shaft 26, the switch 48 is open such that a zero is supplied to the input 54 of inverting amplifier 52 and TRUE is generated at the output 56 of inverting amplifier 52. When the end 42 of the conductor 40 is engaged with the shaft 26, the switch 48 is closed such that battery voltage is applied to the input 54 of inverting amplifier 52 and FALSE is generated at the output 56 of inverting amplifier 52.

The output of inverting amplifier 52 is coupled to an input 58 of an AND gate 60, the output of which energizes the LED 30 when its inputs 58, 62 are TRUE. Input 62 of the AND gate 60 is coupled through switch 34 such that when switch 34 is closed, the input 62 is at battery 46 voltage. Thus, in the illustrative embodiment, the AND gate 60 energizes the LED 30 when the switch 34 is closed and the switch 48 (between the conductor 40 and the shaft 26) is open. In other words, the LED 30 is illuminated when the switch 34 is closed and the end 42 of the conductor 40 is spaced apart from the shaft 26.

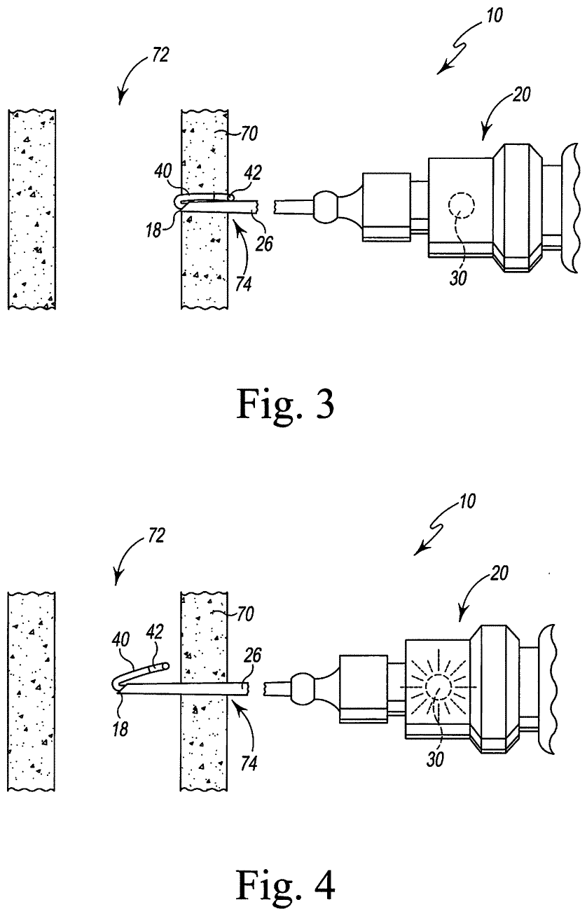

Referring now to FIGS. 3-4, some of the steps of a surgical procedure using the surgical instrument 10 are illustrated. In use, the needle tip 18 of the surgical instrument 10 may be used to form the puncture 74. With the needle tip 18 against the neck of the patient, the switch 34 is closed, turning on the LED 30. The needle tip 18 then punctures the neck. As the needle tip 18 is advanced through the neck, the tissue surrounding the needle shaft 26 causes the end 42 of the conductor 40 to engage the shaft 26, thereby closing the switch 48 and deenergizing the LED 30 as illustrated in FIG. 3.

When the needle tip 18 reaches, and protrudes into, the patient's lumen 72 (e.g., trachea, esophagus, or spinal column), the end 42 of the conductor 40 moves away from the shaft 26, as illustrated in FIG. 4. When the conductor end 42 moves away from the shaft 26, the switch 48 opens and power is again supplied to the LED 30. The LED 30 thereby provides a visual indication to the user that the instrument 10 has penetrated the patient's neck and is in the lumen 72. As noted above, in other embodiments the surgical instrument 10 may additionally or alternatively include an audible indicator to notify the user that the instrument 10 has penetrated the tissue of the patient. In response to receiving the indication that the puncture has been completed, the physician will typically withdraw the needle tip 18 from the tissue.

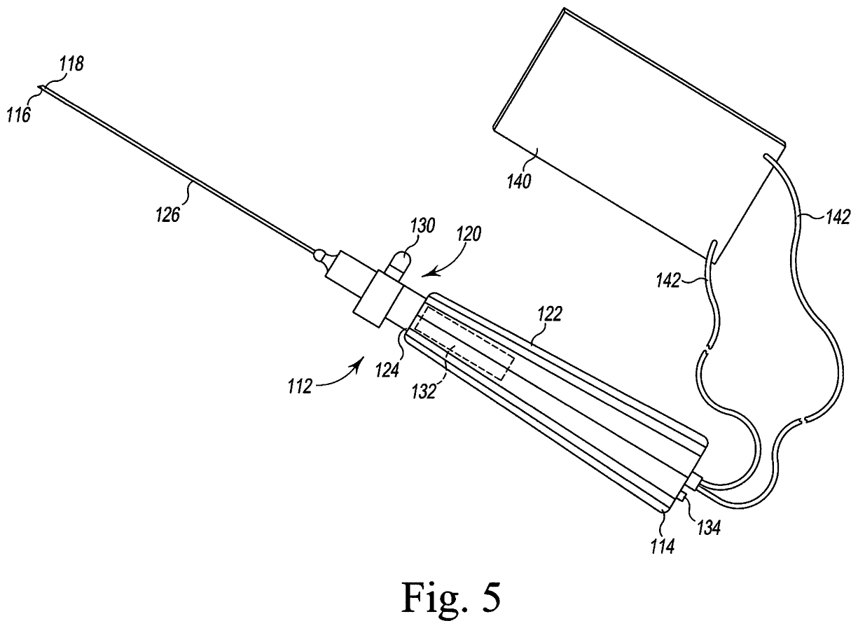

Referring now to FIGS. 5-8, another surgical instrument system including a puncture instrument 110 configured for insertion into the soft tissue of a patient is illustrated. The puncture instrument 110 includes an elongated body 112 that extends from a proximal end 114 to a distal end 116. A needle tip 118 configured to pierce the tissue is formed at the distal end 116 of the body 112. The surgical instrument 110 includes an indicator 120 configured to notify a user that the needle tip 118 has penetrated the tissue.

The elongated body 112 includes a handle 122 extending from the proximal end 114 to a distal handle end 124. A shaft 126 extends from the handle 122 to the needle tip 118. In the illustrative embodiment, the shaft 126 is a cannula formed from an electrically conductive material. The needle tip 118 and the shaft 126 are integrally formed, but it should be appreciated that in other embodiments the needle tip 118 and the shaft 126 may be formed as separate components that are assembled.

As illustrated in FIG. 5, the indicator 120 is positioned at the distal handle end 124. In the illustrative embodiment, the indicator 120 includes a light source such as, for example, an LED 130, and other electrical circuitry 132 operable to energize the LED 130 to provide a visual output to the user. In other embodiments, the indicator 120 may include other electrical circuitry to provide an audible output to the user. The surgical instrument 110 also includes a power switch 134 positioned at the proximal end 114 of the handle 122. Switch 134 is operable to supply power to the electrical circuitry 132 including LED 130.

The surgical instrument 110 also includes a target body 140 configured to be positioned in an internal lumen, for example, the esophagus, of the patient. The target body 140 comprises an electrically conductive material and is configured to be accommodated by the lumen. The target body 140 is coupled to the electrical circuitry 132 by a pair of electrical conductors 142. Illustratively, target body 140 comprises a puncture-resistant, electrically conductive balloon or the like.

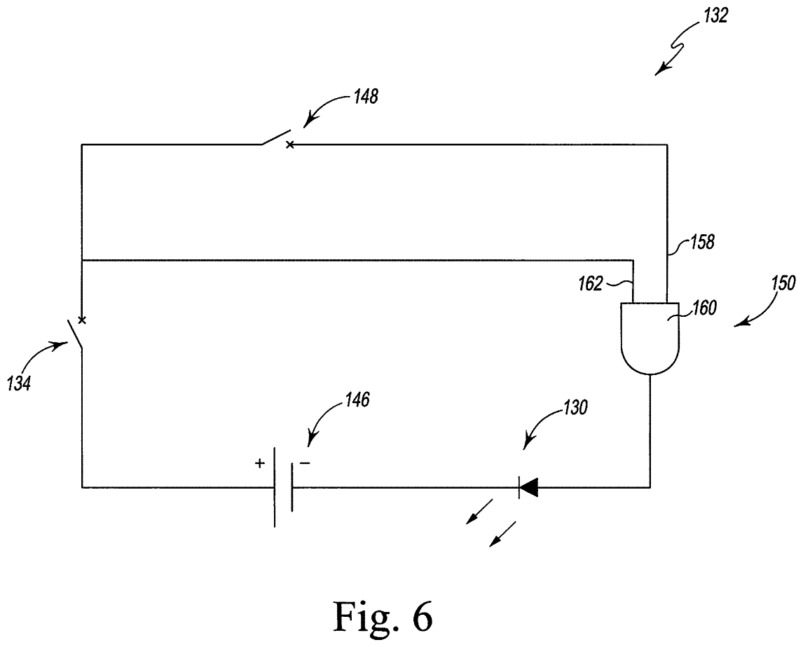

Referring now to FIG. 6, the electrical circuitry 132 includes a battery 146 configured to supply electrical power when the power switch 134 is closed. In the illustrative embodiment, the target body 140 and the needle tip 118 of the shaft 126 comprise the electrical contacts of an electrical switch 148 between switch 134 and a logic circuit 150 operable to energize LED 130. The logic circuit 150 is illustratively embodied as an AND gate, but the logic circuit 150 may be realized as other circuitry as appropriate.

Illustratively, the logic circuit 150 include a single AND gate 160 configured to energize the LED 30 when the inputs 158, 162 of AND gate 160 are high. As illustrated in FIG. 6, the input 158 of the AND gate 160 is coupled to the output of the switch 148 (comprising the target body 140 and the needle tip 118), and the input 162 of the AND gate 160 is coupled to the output of the switch 134. Thus, when both switches 134, 148 are closed, the AND gate 160 energizes the LED 130.

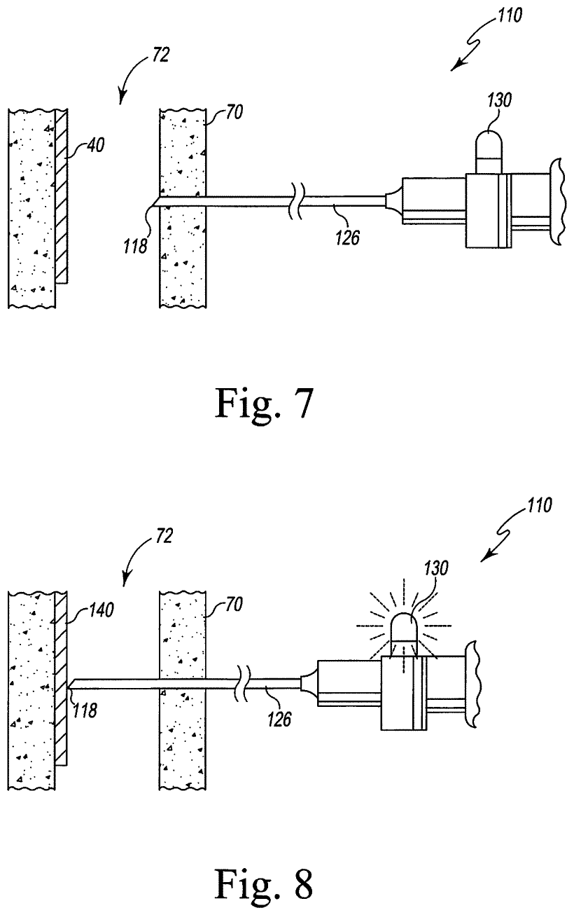

Referring now to FIGS. 7-8, some of the steps of a surgical procedure using the surgical instrument 110 are illustrated. In use, the needle tip 118 of the surgical instrument 110 may be used to form a puncture in, for example, the tracheoesophageal wall 70. Illustrated in FIG. 7, the target body 140 is introduced into the esophagus 72 to the anticipated level at which the needle tip 118 will form the puncture. With the needle tip 118 spaced apart from the tracheoesophageal wall 70, the switch 134 is closed, and the puncture commenced. The LED 130 remains deenergized because the switch 148 comprising tip 118 and target body 140 is not yet closed.

The needle tip 118 exits the tracheoesophageal wall 70 into the esophagus 72 and advances into contact with the target body 140, as illustrated in FIG. 8. When the needle tip 118 contacts the target body 140, the switch 148 closes and power is supplied to the LED 130. A visual indication to the user that the instrument 110 has penetrated the tissue of the patient and is positioned in the esophagus 72 results. As described above, in other embodiments, the surgical instrument may alternatively include an audible indicator to notify the user that the instrument has penetrated the soft tissue of the patient and is positioned in the esophagus 72. In still other embodiments, the surgical instrument may include both a visual indicator and an audible indicator. In response to receiving the indication from the LED 130, the user may withdraw the needle tip 118 from the patient's tissue.

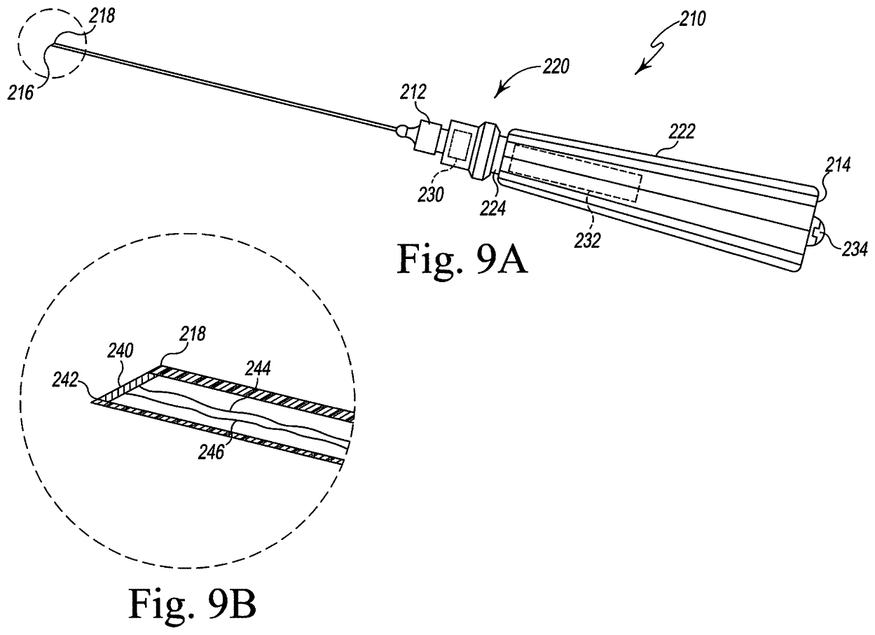

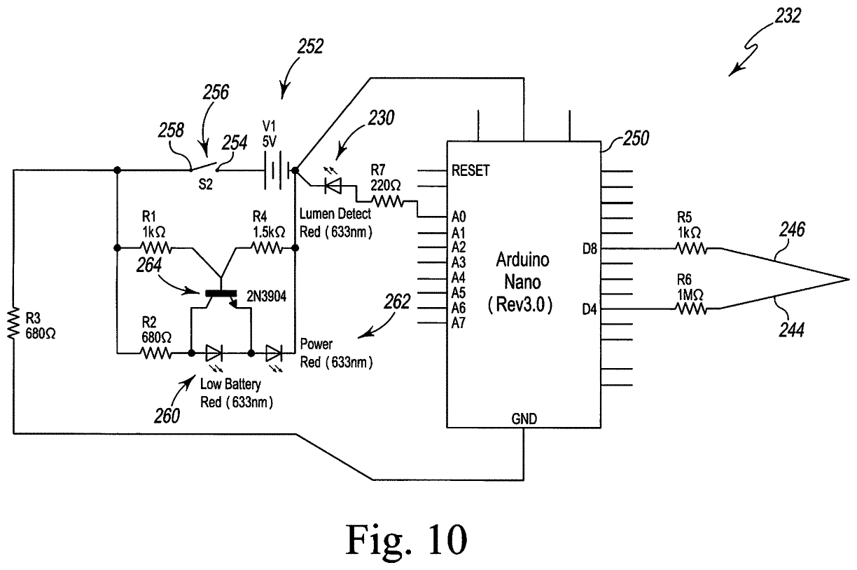

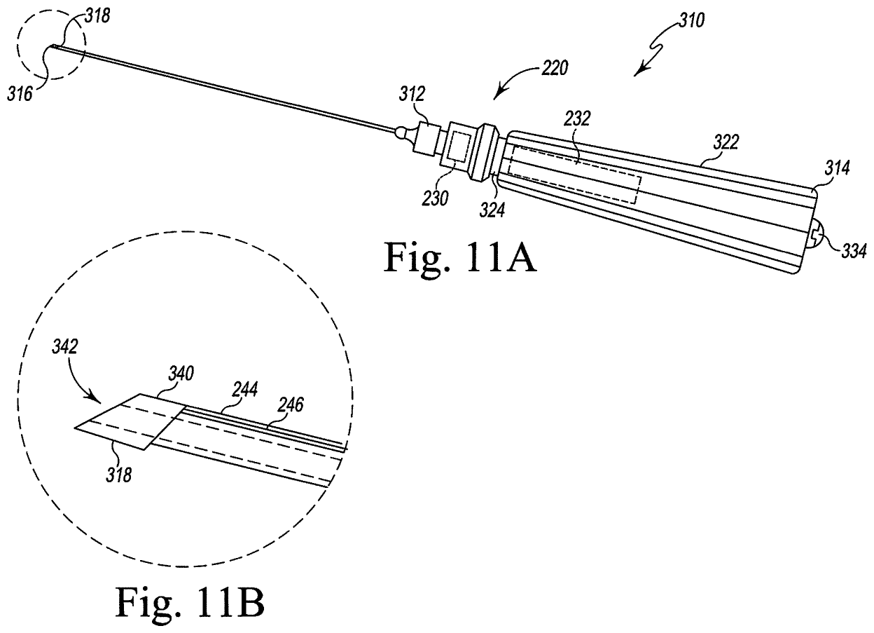

Referring now to FIGS. 9-10, another surgical instrument system including a puncture instrument 210 configured for insertion into the soft tissue of a patient is illustrated. The surgical instrument 210 includes an elongated body 212 that extends from a proximal end 214 to a distal end 216. A needle tip 218 configured to pierce the tissue is formed at the distal end 216 of the body 212. The surgical instrument 210 includes an indicator 220 configured to notify a user that the needle tip 218 has penetrated the tissue. In the illustrative embodiment, the surgical instrument 210 detects increases and decreases in capacitance and will activate the indicator 220 when the capacitance is greater than a predetermined threshold corresponding to the capacitance associated with a human body, as described in greater detail below.

The elongated body 212 includes a handle 222 extending from the proximal end 214 to a distal handle end 224. A shaft 226 extends from the handle 222 to the needle tip 218. In the illustrative embodiment, the shaft 226 is a cannula formed from a non-conductive material such as, for example, a non-conductive ceramic or plastic material. The needle tip 218 and the shaft 226 are integrally formed, but it should be appreciated that in other embodiments the needle tip 218 and the shaft 226 may be formed as separate components that are assembled.

As illustrated in FIG. 9A, the indicator 220 is positioned at the distal handle end 224. In the illustrative embodiment, the indicator 220 includes a light source such as, for example, an LED 230, and other electrical circuitry 232 operable to energize the LED 230 to provide a visual output to the user. In other embodiments, the indicator 220 may include other electrical circuitry to provide an audible output to the user. The surgical instrument 210 also includes a power switch 234 positioned at the proximal end 214 of the handle 222. Switch 234 is operable to supply power to the electrical circuitry 232 including LED 230.

As illustrated in FIG. 9B, the electrical circuitry 232 also includes a conductor plate 240 that is positioned in the distal opening 242 of the needle tip 218. In the illustrative embodiment, the plate 240 covers the opening 242 such that fluid is prevented from entering the needle tip 218. A pair of wires 244, 246 connects the backside of the plate 240 to the electrical circuitry 232. When a patient's tissue contacts the conductor plate 240, the electrical circuitry 232 is operable to detect the electrical capacitance of the tissue, as described in greater detail below.

Referring now to FIG. 10, the electrical circuitry 232 is shown in greater detail. The schematic and block circuit diagram descriptions that follow identify specific integrated circuits and other components of the circuitry 232 and in many cases specific sources for these. Specific terminal and pin names and numbers are generally given in connection with these for the purposes of completeness. It is to be understood that these terminal and pin identifiers are provided for these specifically identified components. It is to be understood that this does not constitute a representation, nor should any such representation be inferred, that the specific components, component values or sources are the only components available from the same or any other sources capable of performing the necessary functions. It is further to be understood that other suitable components available from the same or different sources may not use the same terminal/pin identifiers as those provided in this description.

The circuitry 232 includes a capacitance monitoring circuit 250, for example, an Arduino Nano (rev. 3.0) available from Arduino. A voltage supply includes a 5 VDC battery 252, the anode of which is coupled to one terminal 254 of a switch 256. The other terminal 258 of switch 256 is coupled through a 680Ω resistor to the GrouND terminal of circuit 250. The 5V terminal of circuit 250 is coupled to the cathode of battery 252. Terminal 258 of switch 256 is coupled through a 680Ω resistor to the anode of a red (633 nm) “Low Battery” LED 260. The cathode of LED 260 is coupled to the anode of a red “Power” LED 262. The cathode of LED 262 is coupled to the cathode of battery 252. A series voltage divider of a 1 KΩ resistor and a 1.5 KΩ resistor is coupled across terminal 258 and the cathode of battery 252. The junction of the 1 KΩ resistor and the 1.5 KΩ resistor is coupled to the base of a transistor 264, illustratively, a 2N3904 transistor. The collector of transistor 264 is coupled to the anode of LED 260. The emitter of transistor 264 is coupled to the cathode of LED 260. Thus, as long as transistor 264 is “on,” LED 260 is deenergized. When the voltage on the base of the transistor 264 drops below that required to hold it “on,” it turns off and LED 260 is energized, indicating low battery 252 voltage.

The conductor plate 240 of the needle tip 218 is coupled via the wire 244 through a 1 MΩ resistor to the D4 terminal of circuit 250. The conductor plate 240 is also coupled via the wire 246 through a 1 KΩ resistor to the D8 terminal of the circuit 250. The A0 terminal of circuit 250 is coupled through a 220Ω resistor to the anode of a red “Lumen Detect” LED 230. The cathode of LED 230 is coupled to the cathode of battery 252. When the capacitance increases sharply, such as, for example, when the conductor plate 240 is placed in contact with a patient's tissue, the circuit 250 is programmed to switch the A0 terminal “high,” thereby turning LED 230 “on.” This indicates to the user that the needle tip 218 is engaged with the patient's tissue.

In use, the needle tip 218 of the surgical instrument 210 may be used to form a puncture in the patient's tissue. For example, with the needle tip 218 against the neck of the patient, the conductor plate 240 is engaged with the patient's tissue, and the capacitance increases sharply. The circuit 250 switches the A0 terminal “high,” thereby turning LED 230 “on.” The needle tip 218 then punctures the neck. As the needle tip 218 is advanced through the neck, the conductor plate 240 remains engaged with the patient's tissue and the LED 230 remains energized. When needle tip 218 reaches, and protrudes into, a target lumen of a patient such as, for example, the trachea, esophagus, or spinal column, the conductor plate 240 is no longer in contact with the patient's tissue, thereby causing the capacitance to decrease sharply, and the circuit 250 switches the A0 terminal “low,” thereby turning LED 230 “off.” This indicates to the user that the needle tip 218 is positioned in the target lumen.

The surgical instrument of FIGS. 9-10 utilizes the stored electrical charge or capacitance of the human body to detect when the needle is engaged with a patient's tissue. In other embodiments, the surgical instrument may employ an oscillator or other type of tuned-circuit amplifier to produce an AC current (a current that regularly switches its polarity back and forth). The oscillator may be connected to a conductor plate positioned at the needle tip, which also has the ability to store electrical charge thus has its own capacitance. When the conductor plate is engaged with a patient's tissue, the body's capacitance is introduced into the circuit, and the oscillator has to pump charge into a much larger surface area. That causes the oscillator to detune, or change frequency. This change can be monitored and used to toggle the indicator on or off.

Referring now to FIGS. 11A-B, another surgical instrument system including a puncture instrument 310 that uses the patient's capacitance to provide an indication to the user is shown. The surgical instrument 310 includes an elongated body 312 that extends from a proximal end 314 to a distal end 316. A needle tip 318 configured to pierce the tissue is formed at the distal end 316 of the body 312. The surgical instrument 310 includes an indicator 220 configured to notify a user that the needle tip 318 has penetrated the tissue. In the illustrative embodiment, the surgical instrument 310 detects increases and decreases in capacitance and will activate the indicator 220 when the capacitance is greater than a predetermined threshold corresponding to the capacitance associated with a human body.

The elongated body 312 includes a handle 322 extending from the proximal end 314 to a distal handle end 324. A shaft 326 extends from the handle 322 to the needle tip 318. In the illustrative embodiment, the shaft 326 is a cannula formed from a non-conductive material. The needle tip 318 and the shaft 326 are integrally formed, but it should be appreciated that in other embodiments the needle tip 318 and the shaft 326 may be formed as separate components that are assembled.

As illustrated in FIG. 11A, the indicator 220 is positioned at the distal handle end 324. In the illustrative embodiment, the indicator 220 includes a light source such as, for example, an LED 230, and other electrical circuitry 232 operable to energize the LED 230 to provide a visual output to the user. In other embodiments, the indicator 220 may include other electrical circuitry to provide an audible output to the user. The surgical instrument 310 also includes a power switch 334 positioned at the proximal end 314 of the handle 322. Switch 334 is operable to supply power to the electrical circuitry 332 including LED 330.

As illustrated in FIG. 11B, the electrical circuitry 232 also includes a conductor sheath 340 that is positioned over the needle tip 318. In the illustrative embodiment, the sheath 340 does not cover the opening 342 of the needle tip 318 such that fluid is permitted to enter the needle tip 318. A pair of wires 244, 246 connects the sheath 340 to the electrical circuitry 232. When a patient's tissue contacts the conductor sheath 340, the electrical circuitry 232 is operable to detect the electrical capacitance of the tissue.

The electrical circuitry 232 in the embodiment of FIGS. 11A-B is identical to the electrical circuitry described above in regard to FIGS. 9-10. In use, the needle tip 318 of the surgical instrument 310 may be used to form a puncture in the patient's tissue. With the needle tip 318 against the tissue of the patient, the conductor sheath 340 is engaged with the patient's tissue, and the capacitance increases sharply. The circuit 250 of the electrical circuitry 232 switches the A0 terminal “high,” thereby turning LED 230 “on.” The needle tip 318 then punctures the neck. As the needle tip 318 is advanced through the neck, the conductor sheath 340 remains engaged with the patient's tissue and the LED 230 remains energized. When needle tip 318 reaches, and protrudes into, the target lumen, the capacitance decreases sharply, and the circuit 250 switches the A0 terminal “low,” thereby turning LED 230 “off.” This indicates to the user that the needle tip 318 is positioned in the target lumen of the patient.

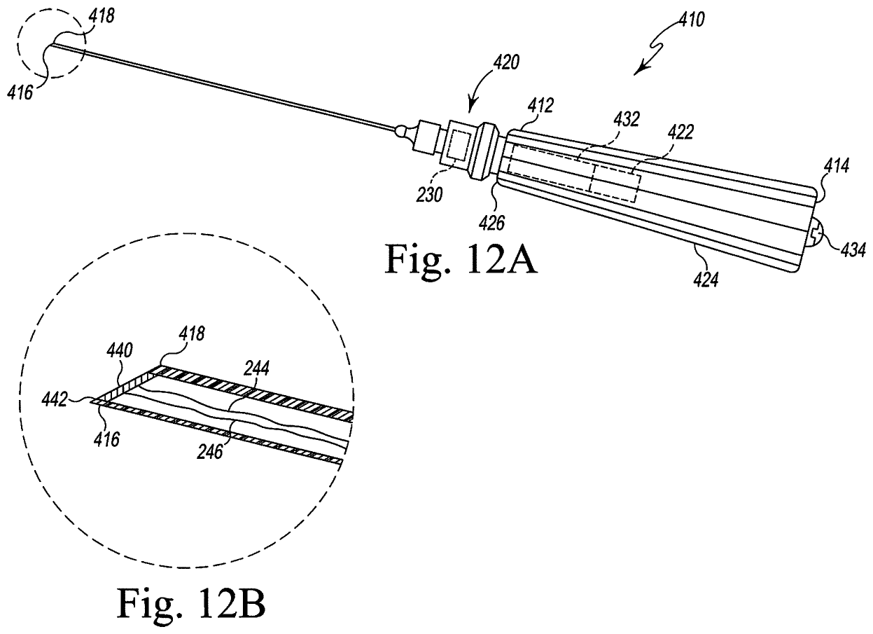

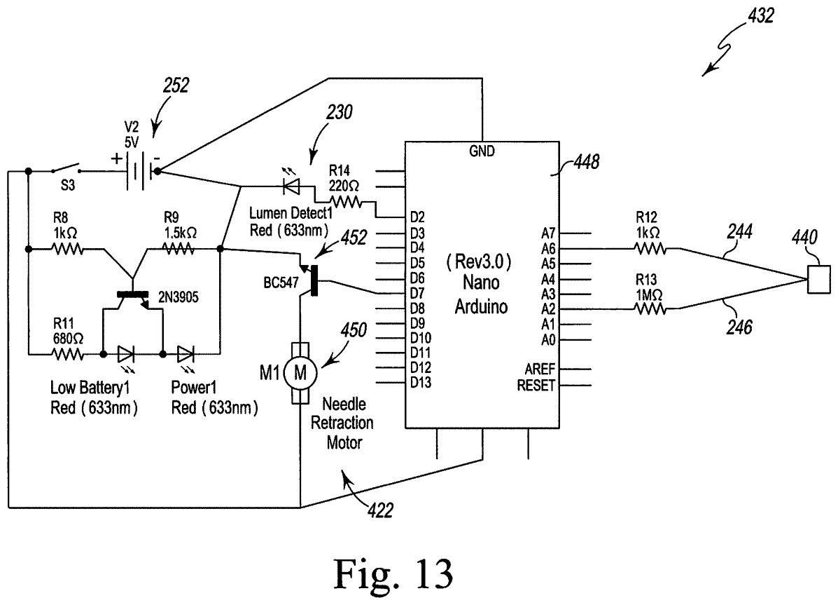

Referring now to FIGS. 12-13, another system including a surgical instrument 410 configured for insertion into the soft tissue of a patient is illustrated. The surgical instrument 410 includes an elongated body 412 that extends from a proximal end 414 to a distal end 416. A needle tip 418 configured to pierce the tissue is formed at the distal end 416 of the body 412. The surgical instrument 410 includes an indicator 420 configured to notify a user that the needle tip 418 has penetrated the tissue and an automatic needle retraction mechanism 422 operable to retract the needle tip 418 after it has penetrated the tissue, as described in greater detail below. In the illustrative embodiment, the surgical instrument 410 detects increases and decreases in capacitance and will activate the indicator 420 when the capacitance is greater than a predetermined threshold corresponding to the capacitance associated with a human body, as described in greater detail below.

The elongated body 412 includes a handle 424 extending from the proximal end 414 to a distal handle end 426. A shaft 428 extends from the handle 424 to the needle tip 418. In the illustrative embodiment, the shaft 428 is a cannula formed from a non-conductive material. The needle tip 418 and the shaft 428 are integrally formed, but it should be appreciated that in other embodiments the needle tip 418 and the shaft 428 may be formed as separate components that are assembled.

As illustrated in FIG. 12A, the indicator 420 is positioned at the distal handle end 426. In the illustrative embodiment, the indicator 420 includes a light source such as, for example, an LED 230, and other electrical circuitry 432 operable to energize the LED 230 to provide a visual output to the user. In other embodiments, the indicator 420 may include other electrical circuitry to provide an audible output to the user. The surgical instrument 410 also includes a power switch 434 positioned at the proximal end 414 of the handle 424. Switch 434 is operable to supply power to the electrical circuitry 432 including LED 230.

As illustrated in FIG. 12B, the electrical circuitry 432 also includes a conductor plate 440 that is positioned in the distal opening 442 of the needle tip 418. In the illustrative embodiment, the plate 440 covers the opening 442 such that fluid is prevented from entering the needle tip 418. A pair of wires 244, 246 connects the backside of the plate 440 to the electrical circuitry 432. When a patient's tissue contacts the conductor plate 440, the electrical circuitry 432 is operable to detect the electrical capacitance of the tissue, as described in greater detail below.

Referring now to FIG. 13, the electrical circuitry 432 is shown in greater detail. Except as otherwise discussed in the description that follows, the circuitry 432 illustrated in FIG. 13 is identical to the circuitry 232 illustrated in FIG. 10. The electrical circuitry 432 includes a circuit 448 that is operable to activate the indicator 420 and the automatic needle retraction mechanism 422. Like the circuit 250 described above, the circuit 448 is illustratively an Arduino Nano (rev. 3.0) circuit. As shown in FIG. 13, the automatic needle retraction mechanism 422 illustratively includes a DC needle retraction motor 450 that is coupled to the shaft 428 (and hence the needle tip 418). The motor 450 is positioned in the handle 424 of the surgical instrument 410 and may be, for example, an Uxcell 3 VDC micromotor. It should be appreciated that the motor 450 may be coupled to the shaft 428 by any appropriate means such as, for example, cam, rack and pinion, etc.

The motor 450 is coupled across the battery 252 of the circuitry 432. The collector-emitter path of a transistor 452, such as, for example, a BC547 transistor, is coupled in series with motor 450 across the battery 252. The base of transistor 452 is coupled to the D7 terminal of the circuit 448. The motor 450 is thus controlled by the signal on the D7 terminal of circuit 448.

The circuitry 432 also includes the LED 230, which is coupled to the cathode of battery 252 and to the D2 terminal of circuit 448 through a 220Ω resistor. In this embodiment, when the capacitance sensed by conductor plate 440 experiences a “step” change, indicating, for example, that the needle tip 418 has penetrated a target lumen such as the trachea, the circuit 432 is programmed to switch the D2 terminal “high,” thereby turning LED 230 “on.” Additionally, the circuit 432 is programmed to switch the terminal D7 “high,” energizing the motor 450 to retract the shaft 428 to reduce the likelihood of damage to the opposite wall of the trachea and to tissue beyond the opposite wall of the trachea.

In use, the needle tip 418 of the surgical instrument 410 may be used to form a puncture in the patient's tissue. As the needle tip 418 is advanced through the neck, the conductor plate 440 is engaged with the patient's tissue. When needle tip 418 reaches, and protrudes into, the target lumen of the patient, the conductor plate 440 is no longer in contact with the patient's tissue, thereby causing the capacitance to decrease sharply, and the circuit 448 switches the D2 terminal and D7 terminal “high,” thereby turning LED 230 “on” and energizing the motor 450 to retract the needle tip 418. In the illustrative embodiment, the needle tip 418 is retracted about 2-3 millimeters. After a predetermined period of time has elapsed, the motor 450 may be deenergized. A spring or other biasing element may be used to urge the needle tip 418 back into its forward position.

In other embodiments, the puncture instrument may be configured to locate and to differentiate between different masses (size and density) in the patient's body since different tissues have different capacitance signatures depending on their mass and density. For example, the puncture instrument may be configured to locate and/or differentiate between different organs and cancerous tissue such that the instrument may be used as probe or biopsy needle. In such embodiments, the instrument may activate a visual or audible in the instrument to alert the operator that the tip has exited one type of tissue and entered a different type of tissue.

In other embodiments, the puncture instrument may be configured to differentiate between good and bad fruit/vegetable based on its density. In such embodiments, the ripe fruit would have a different signature from that of an unripe or spoiled fruit/vegetable.

In other embodiments, a surgical instrument used to form a puncture in the patient's tissue may include a fiber optical thermometer to provide an indication to the user of the location of the needle tip. A fiber optical thermometer needle sensor can be used in electromagnetically strongly influenced environment, in microwave fields, power plants or explosion-proof areas and wherever measurement with electrical temperature sensors is not possible. The thermometer can have 1-255 channels for temperature measurement.

The fiber optical thermometer needle sensor typically consists of a (GaAs) semiconductor crystal that is mounted on the end of an optical fiber. Gallium arsenide (GaAs) is a compound of the elements gallium and arsenic. The needle tip and shaft may be completely non-metallic. The fiber optical sensor may be completely non-conductive and offers complete immunity to RFI, EMI, NMR and microwave radiation with high temperature operating capability, intrinsic safety, and non-invasive use.

The principle of operation is based on the temperature dependence of the band gap of GaAs. The GaAs crystal fixed on the tip of the fiber will be transparent at a wavelength above 850 nm. The position of the band edge is temperature dependent and is shifted about 0.4 nm/Kelvin. The light is directed via the optical fiber to the crystal, where it is absorbed and partially reflected back into the fiber. A miniature spectrometer provides a spectrum with the position of the band edge, from which the temperature is calculated. As the needle travels through different materials, it would give continuous feedback as to the temperature of the material that it is traveling through or when it enters a lumen or an inside space.

In other embodiments, a surgical instrument used to form a puncture in the patient's tissue may include an electromagnetic sensor to provide an indication to the user of the location of the needle tip. Electromagnetic fields, radio waves, microwaves and wireless signals are collectively referred to as radio frequency (RF) energy. Electromagnetic waves are measured by wavelength and frequency. Wavelength is the distance covered by one complete cycle of the electromagnetic wave. Frequency is the number of electromagnetic waves in one second, also known as a hertz or Hz. One Hz equals one cycle per second. One megahertz (MHz) equals one million cycles per second.

The needle can be used to transmit or receive electromagnetic waves and depending on the density of the material that the needle is traveling through the strength of the waves being transmitted or received will very. The material that the needle is traveling through insulates the electromagnetic waves and thus materials of different insulating factors will directly influence the strength of the radio waves as they pass through them.

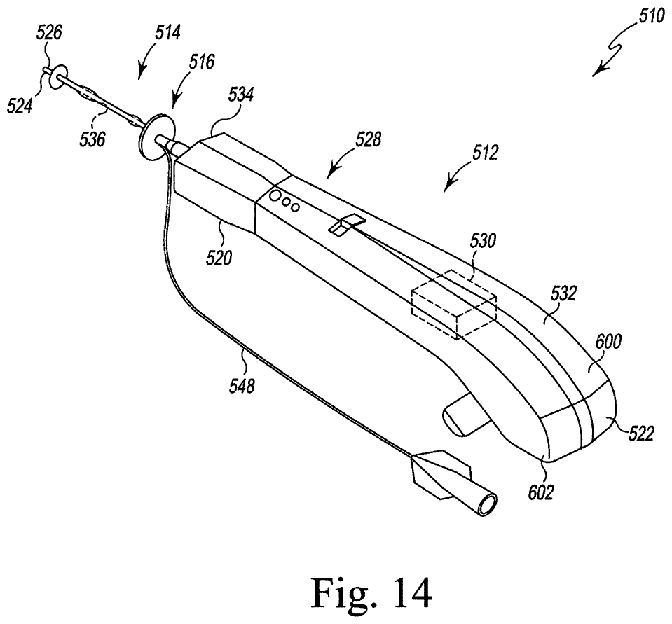

Referring now to FIG. 14, an instrument system 510 for forming and dilating an opening in a patient's tissue is shown. The instrument system 510 includes a puncture instrument 512 similar to the puncture instruments described above in regard to FIGS. 1-13 and a balloon catheter 514 that is removably coupled to the puncture instrument. The system 510 also includes a moveable retainer 516 that is positionable on the balloon catheter 514 to inhibit movement of the balloon catheter 514 during a surgical procedure, as described in greater detail below. The instrument system 510 may be used, for example, to create a puncture or incision in a tracheal wall of a patient and dilate the incision to receive a prosthesis such as, for example, a tracheostomy tube to form an air passageway for the patient.

Illustratively, the puncture instrument 512 may be used to form a puncture between the skin of the neck and the anterior wall of the trachea of a patient, but it should be appreciated that the puncture instrument 512 may be used to form other punctures, incisions, or openings in the patient's tissue. As shown in FIG. 14, the puncture instrument 512 includes an elongated body 520 having a proximal end 522 and a distal end 524. A needle tip 526 configured to pierce the tissue is formed at the distal end 524 of the body 520. As described in greater detail below, the puncture instrument 512 also includes an indicator 528 configured to notify a user that the needle tip 526 has penetrated the tissue and an automatic needle retraction mechanism 530 operable quickly to retract the needle tip 526 a short distance after the needle tip 526 has penetrated the tissue.

The elongated body 520 includes a handle 532 extending from the proximal end 522 to a distal handle end 534. A shaft 536 extends distally away from the handle 532 to the needle tip 526. In the illustrated embodiment, the shaft 536 is a cannula formed from a metallic material. In other embodiments, the shaft may be formed from a ceramic or plastic material. The needle tip 526 and the shaft 536 are integral, but it should be appreciated that in other embodiments the needle tip 526 and the shaft 536 may be formed as separate components and assembled.

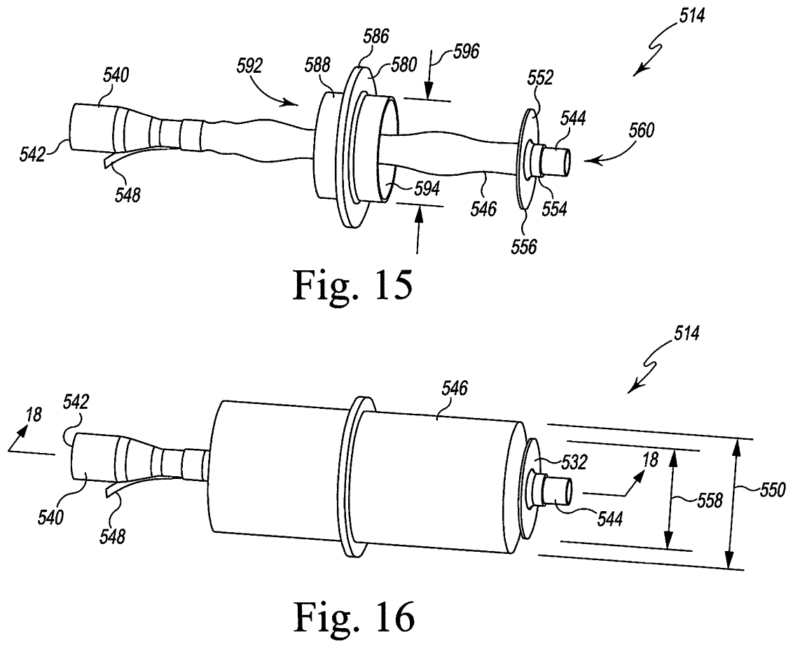

Referring now to FIGS. 15-16, the balloon catheter 514 includes a sheath 540 that extends from a proximal end 542 to a distal end 544. An inflatable balloon 546 extends axially along the sheath 540 between the ends 542, 544. The balloon 546 is illustratively formed from polyethylene terephthalate (“PET”). In other embodiments, the balloon may be formed from nylon, polyurethane, or other non-compliant material. The balloon catheter 514 also includes a tube 548 for the introduction of an inflating fluid into, and exhaust of inflating fluid from, the balloon 546. In the illustrative embodiment, the inflating fluid is water, but it should be appreciated that in other embodiments other suitable fluids may be used. When inflated, the balloon 546 has a generally cylindrical shape, as shown in FIG. 15, having an outer diameter 550 equal to about 0.65 inch (about 16.5 mm) in the illustrative embodiment. The outer diameter 550 is selected based on the desired size of the dilated incision and hence illustratively corresponds to the size of the desired prosthesis. In the illustrative embodiment, the rated pressure of the balloon 546 is about 16 atmospheres.

The balloon catheter 514 also includes a flange 552 that extends outwardly adjacent the distal end 544 of the sheath 540. The flange 552 include a collar 554 that is secured to the sheath 540 and a disk 556 that extends outwardly from the collar 554. The flange 552 is formed as a single, integral component from, for example, PET. In other embodiments, the flange may be formed from nylon, polyurethane, or other non-compliant material. Illustratively, the flange 552 has an outer diameter 558 that is less than the outer diameter 550 of the fully inflated balloon 546 but is greater than the diameter of the completely deflated balloon 546. As described in greater detail below, the outer diameter 558 of the flange 552 is selected such that the flange 552 may initially engage an inner surface of the patient's tissue to resist movement of the balloon catheter 514 during the steps of a surgical procedure. In the illustrative embodiment, the outer diameter 558 is equal to about 0.49 inch (about 12.4 mm). The disk 556 of the flange 552 is sized to deflect or deform relative to the sheath 540 to permit the flange 552 to pass through the opening defined by the puncture instrument 512, as described in greater detail below.

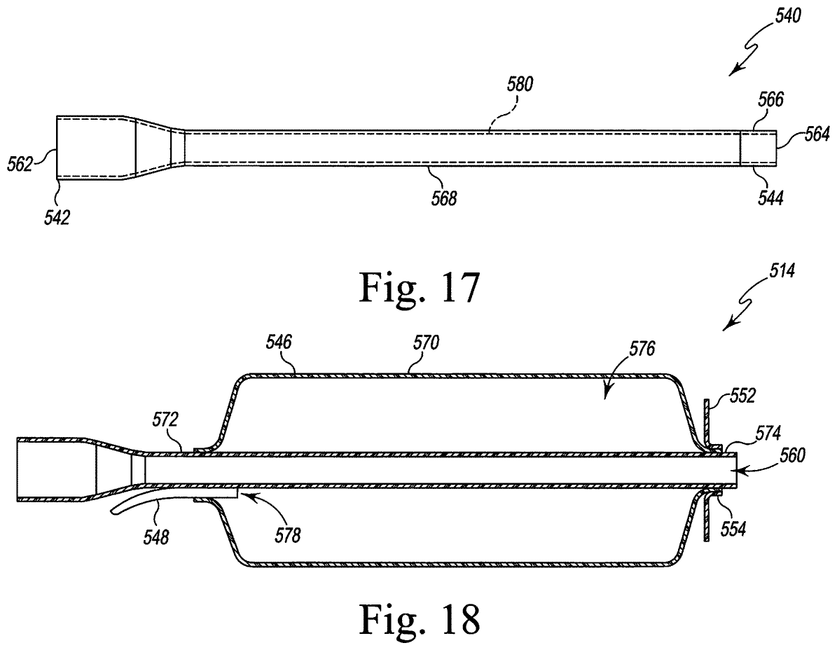

Referring now to FIG. 17, the sheath 540 of the balloon catheter 514 has a lumen 560 extending between an opening 562 at the proximal end 542 and another opening 564 defined the distal end 544. In the illustrative embodiment, the proximal end 542 of the sheath 540 is flared and the lumen 560 is enlarged to receive the needle tip 526 of the puncture instrument 512. The sheath 540 includes a tip 566 at its distal end 544 and an elongated body 568 that extends between the tip 566 and the proximal end 542. In the illustrative embodiment, the tip 566 and the elongated body 568 are formed as separate components that are then assembled to form the sheath 540. In the illustrative embodiment, the tip 566 and the body 568 are formed from plastic materials, but the body 568 is formed from a material that is harder than the tip 566. Illustratively, the tip 566 is formed from polyether block amide (Pebax®) having a hardness of a range of 30 durometer to 50 durometer. The elongated body 68 is also illustratively formed from Pebax® and has a hardness in a range of 70 durometer to 80 durometer. In one illustrative embodiment, the tip 566 has a hardness of 40 durometer, and the body 568 has a hardness of 72 durometer.

Referring now to FIG. 18, the balloon 546 has an inflatable central section 570 formed between a proximal neck 572 and a distal neck 574. The central section 570 defines a chamber 576 into which fluid may be introduced to inflate the central section 570. The distal neck 574 is oriented between the collar 554 of the flange 552 and the sheath 540, and the collar 554 and the neck 574 are bonded to the sheath 540 using an adhesive. The proximal neck 572 is bonded to the elongated body 568 of the sheath 540 also using an adhesive. In the illustrative embodiment, the adhesive is Dymex Adhesive 204-CTH-F. It should be appreciated that in other embodiments other adhesives may be used. As shown in FIG. 18, the tube 548 extends through the proximal neck 572 to an open end 578 oriented in the chamber 576.

Returning to FIG. 15, the system 510 also includes a moveable retainer 516 that is positionable lengthwise on the balloon 546 to resist movement of the balloon catheter 514 during a surgical procedure, as described above. The moveable retainer 516 is illustratively formed as a single, integral component from silicone rubber. It should be appreciated that in other embodiments movable retainer 516 may be formed from other elastomeric materials. Further, in other embodiments, the retainer 516 may be reinforced with a metallic web or frame. In the illustrative embodiment, the silicone rubber has a hardness in the range of 60 and 80 durometer. The retainer 516 has an annular body 580 that extends from a proximal end 582 to a distal end 584. The annular body 580 includes a flange 586 that extends outwardly from a proximal collar 588 and a distal collar 590. A passageway 592 extends from the end 582 to the end 584. The body 580 includes an inner cylindrical surface 594 that defines the passageway 592 and an inner diameter 596. In the illustrative embodiment, the inner diameter 596 is less than the outer diameter 558 of the balloon 546 such that when the balloon 546 is inflated, the retainer 516 fits snuggly onto the balloon 546 and is maintained in its position relative to the balloon 546. Illustratively, the inner diameter 596 of the retainer 516 is about 0.50 inch (about 12.7 mm). In the illustrative embodiment, the flange 586 defines an outer diameter of about 0.80 inch (about 20.3 mm).

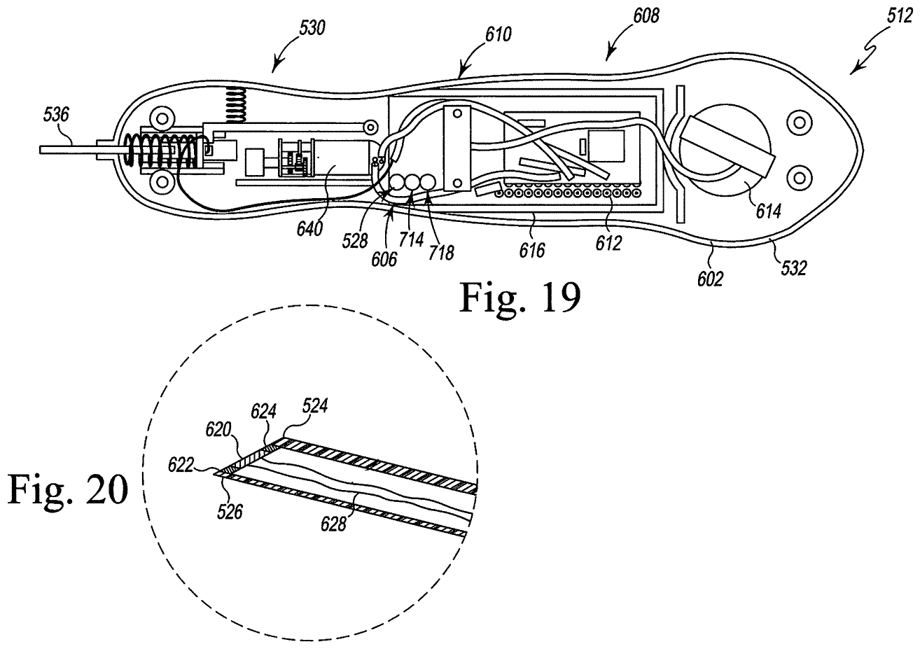

As described above, the dilation system 510 includes a puncture instrument 512 similar to the instruments 10, 210, 310 410 described above. As shown in FIG. 19, the instrument 512 includes a handle 532 and a shaft 536 that extends distally away from the handle 532. The handle 532 illustratively includes an upper housing 600 (see FIG. 14) that is configured to be coupled to a lower housing 602. As illustrated in FIG. 19, the indicator 528 includes a light source such as, for example, a light emitting diode (LED) 606 that is illustratively visible through an opening in the upper housing 600. The housings 600, 602 cooperate to define a chamber in which other electrical circuitry 608 is positioned. The circuitry 608 is operable to energize the LED 606 to provide a visual output to the user. In other embodiments, the indicator 528 may include other electrical circuitry to provide an audible output to the user. The puncture instrument 512 also includes a power switch 610, which is operable to supply power to the electrical circuitry 608 including LED 606.

As illustrated in FIG. 19, the power switch 610 and LED 606 are mounted on a circuit board 612 that is positioned in the handle 532. The circuit board 612 is electrically connected to a battery pack 614 positioned at one end of the handle 532 and the automatic needle retraction mechanism 530, which, as described above, is operable to retract the needle tip 526 a short distance after the needle tip 526 has penetrated the tissue. A metallic plate 616 is positioned below the circuit board 612 between the board 612 and the lower housing 602. In the illustrative embodiment, the plate 616 is formed from copper and is configured to provide a ground plane for the electrical circuitry 608, which makes the user the ground for the electrical circuitry.

As shown in FIG. 20, the electrical circuitry 608 also includes a conductor plate 620 that is positioned in the distal opening 622 of the needle tip 526. In the illustrative embodiment, the plate 620 is electrically insulated from the needle tip 526 by a non-conductive film 624. In other embodiments, the needle tip and/or needle shaft may be formed from a non-conductive material such as, for example, ceramic or plastic to electrically insulate the plate. The plate 620 and the film 624 cooperate to cover the opening 622 such that fluid is prevented from entering the needle tip 526. A wire or conductor 628 connects the backside of the plate 620 to the electrical circuitry 608. When a patient's tissue contacts the conductor plate 620, the electrical circuitry 608 is operable to detect the change in electrical capacitance caused by the contact with the tissue, as described in greater detail below.

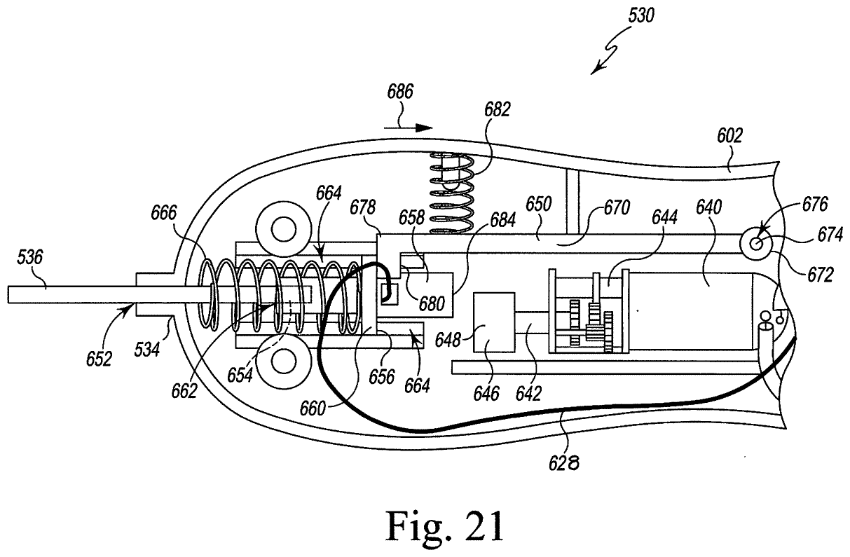

As described above, the instrument 512 includes an automatic needle retraction mechanism 530 operable to retract the needle tip 526 a short distance after the needle tip 526 has penetrated the tissue. As shown in FIG. 21, the needle retraction mechanism 530 includes an electric motor 640 that is connected to an output shaft 642 via a gearbox 644. The needle retraction mechanism 530 also includes a cam 646 that is connected to the output shaft 642. The cam 646 illustratively includes an oblong curved outer surface 648 such that when the cam 646 is rotated by the output shaft 642, the outer surface 648 is moved into and out of engagement with a locking arm 650 that maintains the needle shaft 536 in an extended position.

The needle shaft 536 extends through an opening 652 defined in the distal handle end 534, and the shaft 536 includes a proximal end 654 that is secured to a mounting bracket 656 positioned in the handle 532. The mounting bracket 656 includes a cylindrical body 658 and a slide plate 660 that extends outwardly from the body 658. An aperture 662 is defined at one end of the cylindrical body 658, which receives the proximal end 654 of the shaft 536 and provides a passageway through which the connecting wire 628 passes to connect the conductor plate 620 to the other electrical circuitry 608.

As shown in FIG. 21, the edges of the slide plate 660 are received in a pair of guide slots 664 defined in the handle 532, which guide the movement of the mounting bracket 656 as the needle tip 526 is retracted. A biasing element such as, for example, a spring 666 positioned between the slide plate 660 and the distal handle end 534. In the illustrative embodiment, the spring 666 is configured to bias the slide plate 660 away from the distal handle end 534 and hence bias the needle tip 526 is the retracted position.

The locking arm 650 of the automatic needle retraction mechanism 530 includes an elongated shaft 670 pivotally coupled to the handle 532. A pivot pin 674 extends outwardly from the lower housing 602 and is received in a bore 676 defined in an end 672 of the elongated shaft 670. The elongated shaft 670 extends from the end 672 to an opposite end 678 positioned adjacent the mounting bracket 656. The locking arm 650 includes a tip 680 that extends from the end 678 toward the mounting bracket 656, as illustrated in FIG. 21. The retraction mechanism 530 also includes another biasing element or spring 682 positioned between the elongated shaft 670 and an inner wall of the handle 532.

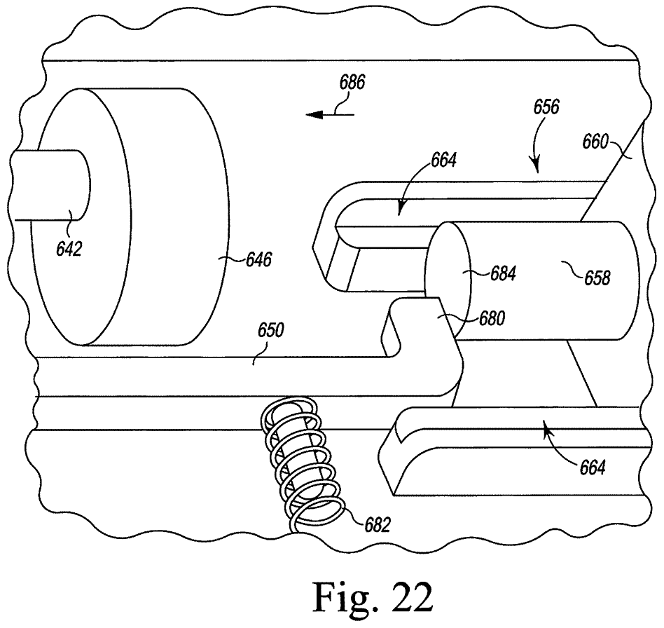

When the needle shaft 536 is in its extended position and ready for insertion into a patient's tissue, the locking arm 650 may initially be engaged with a proximal end 684 of the mounting bracket 656, as shown in FIG. 22. The spring 682 applies a force to the elongated shaft 670 to bias the locking arm 650 in engagement with the proximal end 684, thereby resisting the force exerted by the spring 666 against the slide plate 660 and maintaining the needle shaft 536 in the extended position.

As described above, the automatic needle retraction mechanism 530 is operable to quickly retract the needle tip 526 a short distance after the needle tip 526 has penetrated the tissue. To do so, the motor 640 is energized to rotate the output shaft 642 and the cam 646. As the cam 646 is rotated, its oblong curved outer surface 648 is advanced into engagement with the elongated shaft 670 to move the shaft 670 (and hence the locking arm 650) away from the proximal end 684 of the mounting bracket 656. When the locking arm 650 disengages from the mounting bracket 656, the spring 666 urges the mounting bracket 656 in the direction indicated by arrow 686 in FIGS. 21-22. As the mounting bracket 656 moves, the needle tip 526 retracts away from the opposite wall of the patient's lumen. The spring 666 urges the bracket 656 to continue to move as indicated by arrow 686 until the slide plate 660 is advanced into contact with the locking arm tip 680, as shown in FIG. 21.

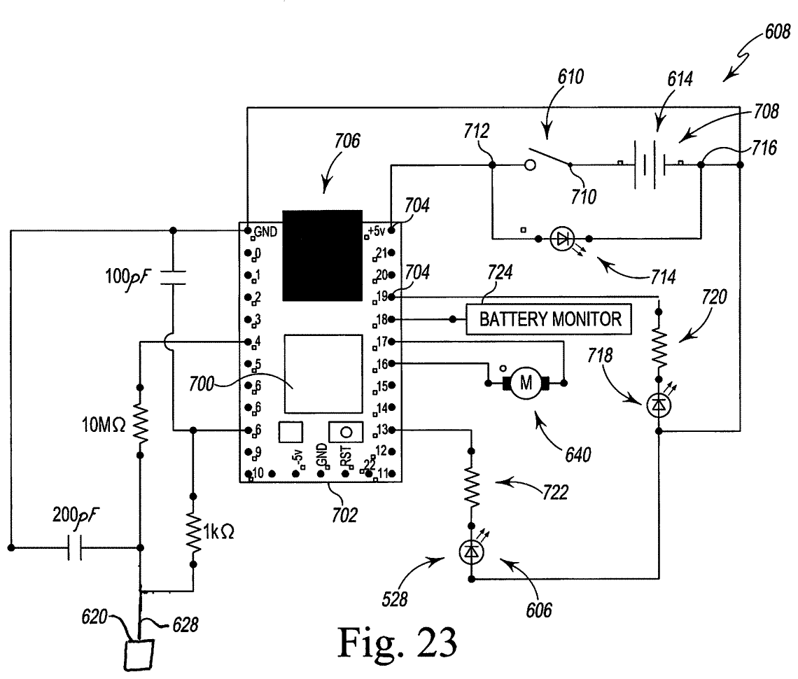

Referring now to FIG. 23, the electrical circuitry 608 is illustrated in greater detail. The schematic and block circuit diagram descriptions that follow identify specific integrated circuits and other components of the circuitry 608 and in many cases specific sources for these. Specific terminal and pin names and numbers are generally given in connection with these for the purposes of completeness. It is to be understood that these terminal and pin identifiers are provided for these specifically identified components. It is to be understood that this does not constitute a representation, nor should any such representation be inferred, that the specific components, component values or sources are the only components available from the same or any other sources capable of performing the necessary functions. It is further to be understood that other suitable components available from the same or different sources may not use the same terminal/pin identifiers as those provided in this description.

The circuitry 608 includes a microprocessor 700 such as, for example, an 8-Bit AVR 16 MHz Processor (ATMEGA32U4) commercially available from Atmel Corporation. As shown in FIG. 23, the microprocessor 700 is attached a circuit 702 that also includes various terminals 704 connected to other circuitry 608. An I/O port 706 such as, for example, a USB port, is attached to the circuit 702 to permit a user to upload software and data to, and download from, the microprocessor 700. Illustratively, the microprocessor 700, the circuit 702, and the I/O port 706 are available in a Teensy 2.0 USB-based microcontroller development system by PJRC. A voltage supply includes two 3 VDC batteries 708 in the battery pack 614, the anode of which is coupled to one terminal 710 of the power switch 610. The other terminal 712 of switch 610 is coupled to the 5V terminal of the circuit 702 and to the anode of a “Power Indicator” LED 714. The cathode of the Power Indicator LED 714 is coupled to the cathode of the battery pack 614 and to the GrouND terminal of the circuit 702 at the terminal 716.

The circuitry 608 also includes a “Low Battery” LED 718, which is energized by the microprocessor 700 when battery voltage drops below a predetermined threshold. The cathode of the LED 718 is connected through a 220Ω resistor 720 to the “19” terminal of the circuit 702. The anode of the LED 718 is connected to the GrouND terminal of the circuit 702 and an anode of the LED 528 of the indicator 606. The cathode of the LED 528 is connected to the “13” terminal of the circuit 702 through another 220Ω resistor 722.

As shown in FIG. 23, the motor 640 is connected to the “16” and “17” terminals, while a battery monitor 724 is connected to the “18” terminal of the circuit 702. The conductor plate 620 of the needle tip 526 is coupled via the wire 628 through a 10Ω resistor to the “4” terminal of the circuit 702. The wire 628 (and hence the “4” terminal) is also connected to the ground terminal of the circuit 702 via a 220 pF capacitor, which assists in stabilizing the circuit. The conductor plate 620 is also coupled via the wire 628 through a 1 KΩ resistor to the “8” terminal of the circuit 620. The wire 628 (and hence the “8” terminal) is connected to the ground terminal of the circuit 702 via a 100 pF capacitor, which also assists in stabilizing the circuit.

Illustratively, when the capacitance sensed by microprocessor 700 through the conductor plate 620 experiences a “step” change, indicating, for example, that the needle tip 526 has penetrated a lumen, the microprocessor 700 is programmed to switch the “13” terminal continuously “high,” thereby turning the indicator LED 528 continuously “on.” Additionally, the microprocessor 700 is programmed to switch the “16” terminal to “high,” thereby energizing the motor 640 to retract the needle tip 526 and reduce the likelihood of damage to the opposite wall of the trachea and to tissue beyond the opposite wall of the trachea.

In use, the needle tip 526 of the puncture instrument 512 may be used to form a puncture in the patient's tissue. To do so, the needle tip 526 may be first inserted into the lumen 560 of the balloon catheter 514 such that the tip 526 extends outwardly from the distal opening 564. When the user toggles the power switch 610, power is supplied to the circuit 702 and to the LED 714, which is energized to indicate that power is “on.” The microprocessor 700 monitors the signal on the “18” terminal from the battery monitor 724. If the microprocessor 700 determines that the voltage signal on the “18” terminal is below a predetermined threshold, the microprocessor 700 is programmed to switch the “19” terminal to “high,” thereby turning the indicator LED 718 “on” to indicate to the user that the battery pack 614 should be replaced.

If the microprocessor 700 determines the voltage signal is above the predetermined threshold, the needle tip 526 may be advanced into contact with the patient's tissue, illustratively the patient's neck tissue. When the tip 526 engages the patient's tissue, the capacitance experienced by conductor plate 620 increases sharply. In the illustrative embodiment, the microprocessor 700 is programmed to consecutively toggle the “13” terminal “high” and “low,” thereby causing the LED 528 to flash “on” and “off” to indicate to the user that the instrument 512 is armed.

As the needle tip 526 is advanced through the neck, the conductor plate 620 remains engaged with the patient's tissue. When needle tip 526 reaches, and protrudes into, the target lumen, the conductor plate 620 is no longer in contact with the patient's tissue, thereby causing the capacitance to decrease sharply, and the microprocessor 700 is programmed to switch the “13” terminal continuously “high,” thereby turning the indicator LED 528 continuously “on.” Additionally, the microprocessor 700 is programmed to switch the “16” terminal to “high,” thereby energizing the motor 640 to retract the needle tip 526. When the motor 640 is energized, the output shaft 642 and hence the cam 646 are rotated. As the cam 646 is rotated, its oblong curved outer surface 648 is advanced into engagement with the elongated shaft 670 to move the shaft 670 (and hence the locking arm 650) away from the proximal end 684 of the mounting bracket 656. When the locking arm 650 disengages from the mounting bracket 656, the spring 666 urges the mounting bracket 656 in the direction indicated by arrow 686 in FIGS. 21-22. As the mounting bracket 656 moves, the needle tip 526 retracts away from the opposite wall of the patient's lumen. The spring 666 urges the bracket 656 to continue to move as indicated by arrow 686 until the slide plate 660 is advanced into contact with the locking arm tip 680, as shown in FIG. 21. In the illustrative embodiment, the needle tip 526 is retracted about 2-3 millimeters.

With the balloon catheter 514 positioned on the puncture instrument 512 and the balloon 546 deflated, the distal tip 566 of the sheath 540 and the flange 552 may be advanced through the incision made by the needle tip 526. As the flange 552 passes through the incision, it may be deflected to decrease its diameter and permit it to enter the incision. Once within the patient's trachea or other lumen, the flange 552 may deflect outward to its normal diameter 558. Because the tip 566 of the sheath 540 is formed from a relatively soft material, it may be advanced into contact with the opposite wall of the patient's lumen without fear of damage. When the flange 552 is positioned in the patient's lumen, the balloon catheter 514 may be pulled outward to advance the flange 552 into contact with an inner surface of the lumen.

With the flange 552 engaged with the inner surface of the trachea, the retainer 516 may be advanced along deflated balloon 546 to engage the outer surface of the trachea opposite the flange 552. In that way, the flange 552 and the retainer 516 cooperate to maintain the balloon 546 in position relative to the patient's tissue and inhibit movement of the balloon 546. The central section 570 of the balloon 546 may then be inflated to dilate the incision to the desired size.

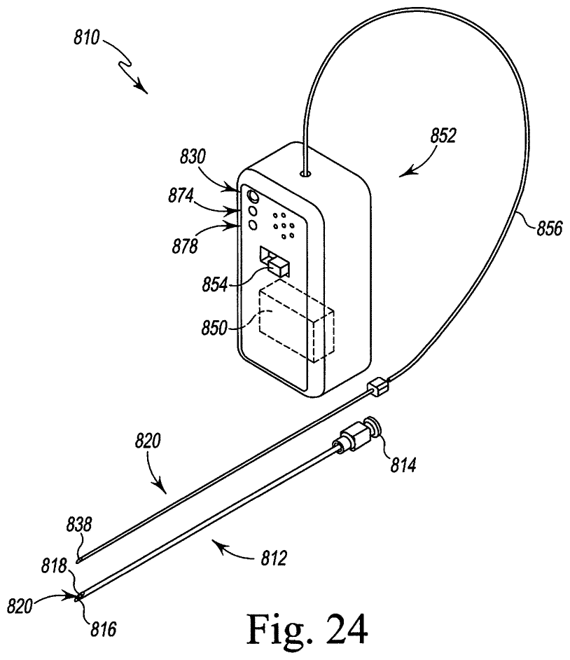

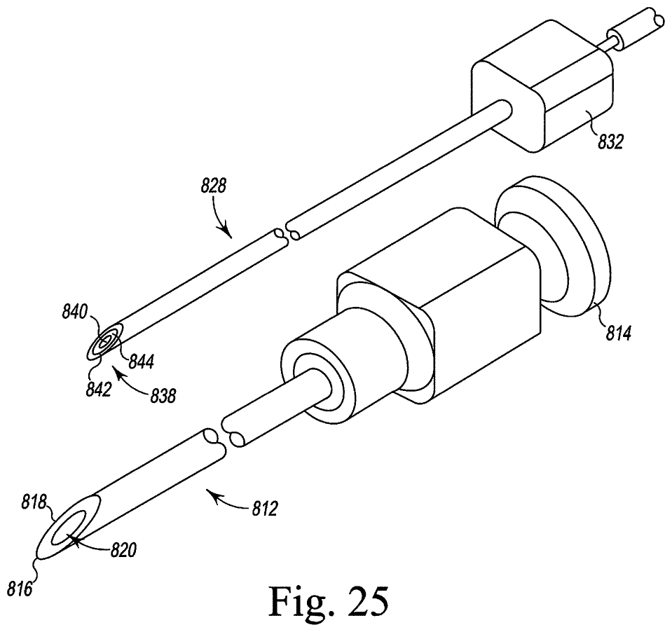

Referring now to FIGS. 24-26, another surgical instrument system 810 configured for insertion into the soft tissue of a patient is illustrated. The surgical instrument system 810 includes an elongated needle body 812 that extends from a proximal end 814 to a distal end 816. A needle tip 818 configured to pierce the tissue is formed at the distal end 816 of the body 812. The needle body 812 has a lumen or passageway 820 extending through the ends 814, 816, as shown in FIG. 25. In the illustrative embodiment, a catheter may be inserted into the passageway 820 to provide, for example, epidural anesthesia, to a patient. The surgical instrument system 810 also includes a probe 828 that is sized to be positioned in the passageway 820 of the needle 812. The probe 828 is connected to an indicator 830 that is configured to notify a user that the needle tip 818 has penetrated the tissue, as described in greater detail below.

The probe 828 includes a base 832 and a shaft 836 that extends distally away from the base 832 to a tip 838. In the illustrated embodiment, the shaft 836 is a cannula formed from an electrically conductive material. The tip 838 and the shaft 836 are integral, but it should be appreciated that in other embodiments the tip 838 and the shaft 836 may be formed as separate components and assembled. As shown in FIG. 25, the probe 828 includes a conductor plate 840 that is positioned in the distal opening 842 of the tip 838. In the illustrative embodiment, the plate 840 is electrically insulated from the tip 838 by a non-conductive film 844. In other embodiments, the shaft may be formed from a non-conductive material such as ceramic or plastic to insulate the plate. The plate 840 and the film 844 cooperate to cover the opening 842 such that fluid is prevented from entering the tip 838. When a patient's tissue contacts the conductor plate 840, electrical circuitry 850 of the system 810 is operable to detect the change in electrical capacitance caused by the contact with the tissue, as described in greater detail below.

Returning to FIG. 24, the system 810 includes a control box 852 that houses the electrical circuitry 850, including the indicator 830. In the illustrative embodiment, the control box 852 has a power switch 854 that may be toggled to energize the electrical circuitry 850. A cable 856 connects the electrical circuitry 850 with the probe 828.

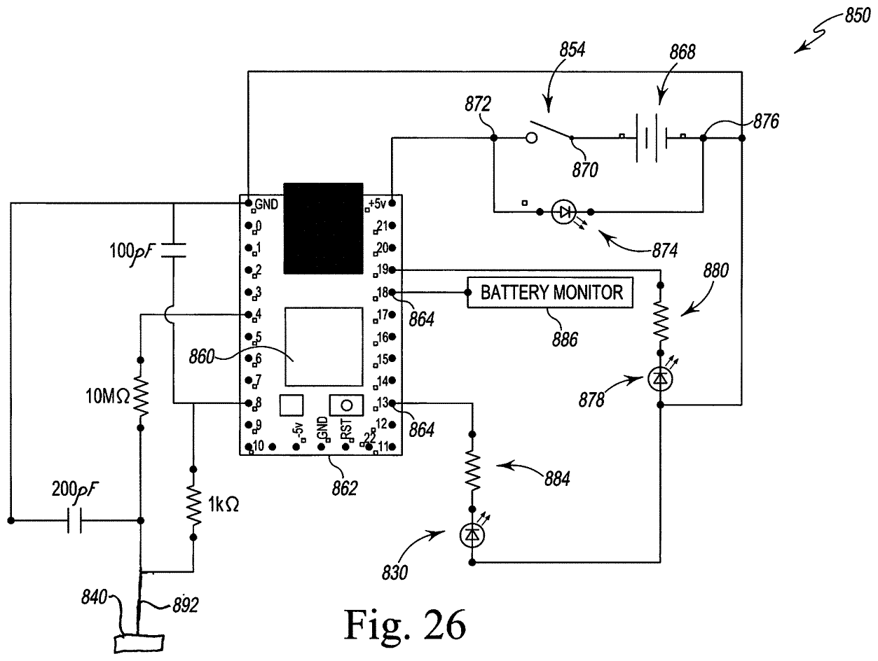

Referring now to FIG. 26, the circuitry 850 includes a microprocessor 860 such as, for example, an 8-Bit AVR 16 MHz Processor (ATMEGA32U4) commercially available from Atmel Corporation. The microprocessor 860 is attached a circuit 862 that also includes various terminals 864 connected to other circuitry 850. An I/O port 866 such as, for example, a USB port, is attached to the circuit 862 to permit a user to upload software and data to, and download from, the microprocessor 860. Illustratively, the microprocessor 700, the circuit 702, and the I/O port 706 are available in a Teensy 2.0 USB-based microcontroller development system. A voltage supply includes two 3 VDC batteries 868, the anodes of which are coupled to one terminal 870 of the power switch 854. The other terminal 872 of switch 854 is coupled to the 5V terminal of the circuit 862 and to the anode of a “Power Indicator” LED 874. The cathode of the Power Indicator LED 874 is coupled to the cathodes of the batteries 868 and to the GrouND terminal of the circuit 862 at the terminal 876.

The circuitry 850 also includes a “Low Battery” LED 878, which is energized by the microprocessor 860 when battery voltage drops below a predetermined threshold. The cathode of the LED 878 is connected through a 220Ω resistor 880 to the “19” terminal of the circuit 862. The anode of the LED 878 is connected to the GrouND terminal of the circuit 862 and an anode of the indicator LED 830. The cathode of the LED 830 is connected to the “13” terminal of the circuit 862 through another 220Ω resistor 884. A battery monitor 886 is connected to the “18” terminal of the circuit 862.

The conductor plate 840 of the tip 838 is coupled via a wire 892 through a 10 MΩ resistor to the “4” terminal of the circuit 862. The wire 892 (and hence the “4” terminal) is also connected to the ground terminal of the circuit 862 via a 220 pF capacitor, which assists in stabilizing the circuit. The conductor plate 840 is also coupled via the wire 892 through a 1 KΩ resistor to the “8” terminal of the circuit 862. The wire 892 (and hence the “8” terminal) is connected to the ground terminal of the circuit 862 via a 100 pF capacitor, which also assists in stabilizing the circuit.

Illustratively, when the capacitance sensed by microprocessor 860 through the conductor plate 840 experiences a “step” change, indicating, for example, that the tip 838 has penetrated a lumen, the microprocessor 860 is programmed to switch the “13” terminal continuously “high,” thereby turning the indicator LED 830 continuously “on.”

In use, the instrument system 810 may be used to form a puncture in the patient's tissue. To do so, the probe 828 may be first inserted into the lumen 820 of the needle 812 such that the probe tip 838 is exposed at the needle tip 818. When the user toggles the power switch 854, power is supplied to the circuitry 850 and to the LED 874, which is energized to indicate that power is “on.” The microprocessor 860 monitors the signal on the “18” terminal from the battery monitor 886. If the microprocessor 860 determines that the voltage signal on the “18” terminal is below a predetermined threshold, the microprocessor 860 is programmed to switch the “19” terminal to “high,” thereby turning the indicator LED 878 “on” to indicate to the user that the batteries 868 should be replaced.

If the microprocessor 860 determines the voltage signal is above the predetermined threshold, the probe tip 838 and needle tip 818 may be advanced into contact with the patient's tissue, illustratively the skin covering a patient's spinal column. When the probe tip 838 engages the patient's tissue, the capacitance experienced by conductor plate 840 increases sharply. In the illustrative embodiment, the microprocessor 860 is programmed to consecutively toggle the “13” terminal “high” and “low,” thereby causing the LED 830 to flash “on” and “off” to indicate to the user that the instrument system 810 is armed. As the needle 812 (and hence the probe 838) is advanced into the spinal column, the conductor plate 840 remains engaged with the patient's tissue.

When the probe tip 838 reaches, and protrudes into, the target lumen (e.g., the interior of the spinal column), the capacitance on the conductor plate 840 decreases sharply, and the microprocessor 860 is programmed to switch the “13” terminal continuously “high,” thereby turning the indicator LED 830 continuously “on” to inform the user to hold the needle 812 in position. The user may then remove the probe 828 from the lumen 820 of the needle 812 while leaving the needle 812 inserted into the patient's tissue. The user may then use the lumen 820 to position, for example, a catheter to provide fluids to the patient.

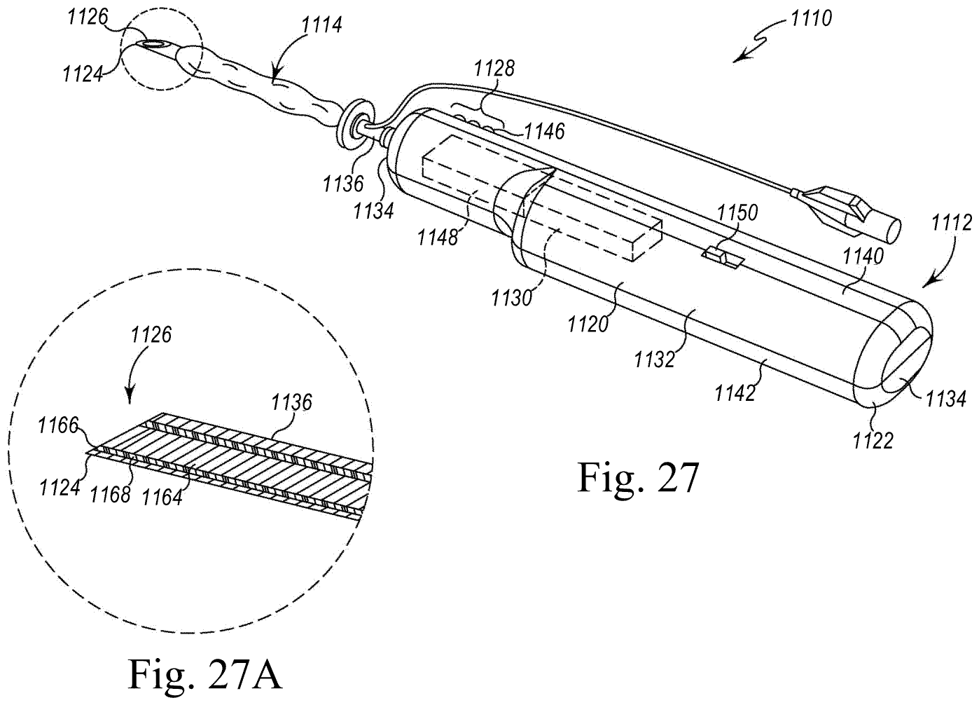

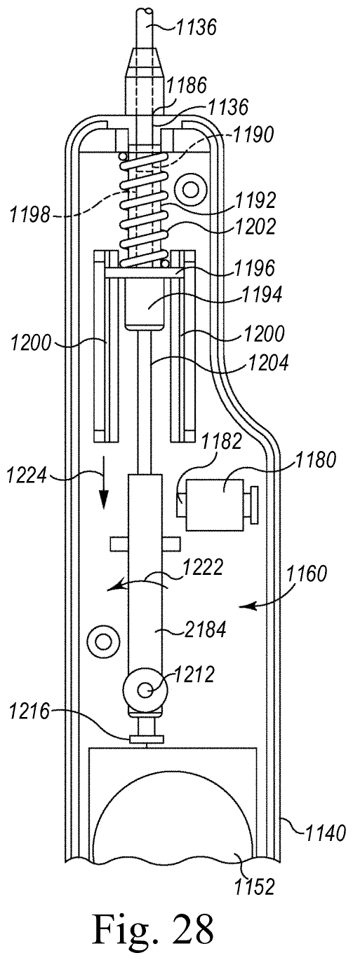

Referring now to FIG. 27, another instrument system 1110 configured for insertion into the soft tissue of a patient is illustrated. The system 1110 is also configured for forming and dilating an opening in a patient's tissue is shown. The instrument system 1110 includes a puncture instrument 1112 and a balloon catheter 1114 that is removably coupled to the puncture instrument. An exemplary balloon catheter for use in the system 1110 is shown and described in U.S. patent application Ser. No. 14/996,426, which is expressly incorporated herein by reference. The instrument system 1110 may be used, for example, to create a puncture or incision in a tracheal wall of a patient and dilate the incision to receive a prosthesis such as, for example, a tracheostomy tube to form an air passageway for the patient. For convenience, the balloon catheter 1114 is not shown in the illustrations of FIGS. 28-33.

Illustratively, the puncture instrument 1112 may be used to form a puncture between the skin of the neck and the anterior wall of the trachea of a patient, but it should be appreciated that the puncture instrument 1112 may be used to form other punctures, incisions, or openings in the patient's tissue. As shown in FIG. 27, the puncture instrument 1112 includes an elongated body 1120 having a proximal end 1122 and a distal end 1124. A needle tip 1126 configured to pierce the tissue is formed at the distal end 1124 of the body 1120. As described in greater detail below, the puncture instrument 1112 also includes an indicator 1128 configured to notify a user that the needle tip 1126 has penetrated the tissue and an automatic needle retraction mechanism 1130 operable quickly to retract the needle tip 1126 a short distance after the needle tip 1126 has penetrated the tissue.

The elongated body 1120 includes a handle 1132 extending from the proximal end 1122 to a distal handle end 1134. A shaft 1136 extends distally away from the handle 1132 to the needle tip 1126. In the illustrated embodiment, the shaft 1136 is a cannula formed from a metallic material. In other embodiments, the shaft may be formed from a ceramic or plastic material. The needle tip 1126 and the shaft 1136 are integral, but it should be appreciated that in other embodiments the needle tip 1126 and the shaft 1136 may be formed as separate components and assembled.

The handle 1132 illustratively includes an upper housing 1140 that is configured to be coupled to a lower housing 1142. The indicator 1128 includes a light source such as, for example, a plurality of light emitting diodes (LED) 1146 that is illustratively visible through an opening in the upper housing 1140. The housings 1140, 1142 cooperate to define a chamber in which other electrical circuitry 1148 is positioned. The circuitry 1148 is operable to energize the LED 1146 to provide a visual output to the user. In other embodiments, the indicator 1128 may include other electrical circuitry to provide an audible output to the user. The puncture instrument 1112 also includes a power switch 1150, which is operable to supply power to the electrical circuitry 1148 including LEDs 1146.

As shown in FIG. 28, the electrical circuitry 1148 includes a battery pack 1152 positioned at one end of the handle 1132 and the automatic needle retraction mechanism 1160, which is operable to retract the needle tip 1126 a short distance after the needle tip 1126 has penetrated the tissue. In illustrative embodiment, the distance is 8 millimeters. A metallic plate (not shown) is positioned in handle 1132 is formed from copper and is configured to provide a ground plane for the electrical circuitry 1148, which makes the user the ground for the electrical circuitry.

Returning to FIG. 27A, the instrument 1112 also includes a conductor plate 1164 that is positioned in the distal opening 1166 of the needle tip 1126. In the illustrative embodiment, the plate 1164 is a metallic shaft that is electrically insulated from the needle tip 1126 by a non-conductive film 1168. In the illustrative embodiment, the film 44 is a cylindrical ring having a predetermined thickness that surrounds the plate 40. In other embodiments, the needle tip and/or needle shaft may be formed from a non-conductive material such as, for example, ceramic or plastic to electrically insulate the plate. The shaft 1164 and the film 1168 cooperate to cover the opening 1166 such that fluid is prevented from entering the needle tip 1126. A wire or conductor 1170 connects the shaft 1164 to the electrical circuitry 1148, and another wire or conductor 1172 connects the outer cannula shaft 1136 to the electrical circuitry 1148. When a patient's tissue contacts the conductor plate 1164, the electrical circuitry 1148 is operable to detect the change in electrical resistance caused by the contact with the tissue, as described in greater detail below.

As described above, the instrument 1112 includes an automatic needle retraction mechanism 1160 operable to retract the needle tip 1126 a short distance after the needle tip 1126 has penetrated the tissue. As shown in FIG. 28, the needle retraction mechanism 1160 includes an actuator 1180. In the illustrative embodiment, the actuator 1180 is a linear actuator such as, for example, a solenoid, which includes an output shaft 1182 operable to move along a straight line. An exemplary actuator is the Uxcell a14092600ux0438 Open Frame Actuator, which is electrically-operated. In other embodiments, the actuator may be embodied as an electric motor, electromagnet, or other electromechanical device operable to move the locking arm 1184, as described in greater detail below. As shown in FIG. 28, the locking arm 1184 that maintains the needle shaft 1136 in an extended position.

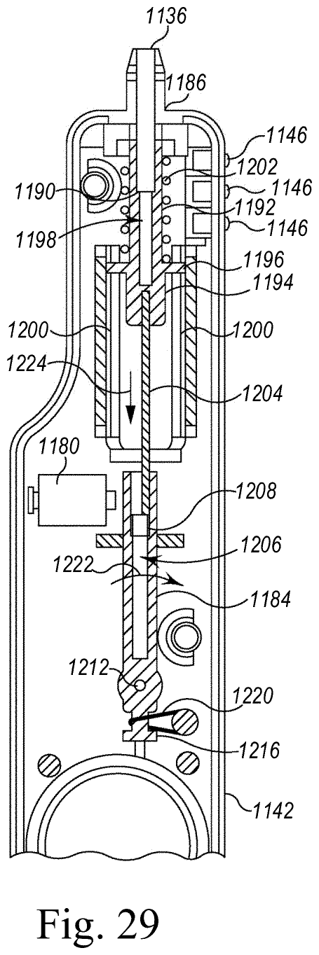

The needle shaft 1136 extends through an opening 1186 defined in the distal handle end 1134, and the shaft 1136 includes a proximal end 1190 that is secured to a mounting bracket 1192 positioned in the handle 1132. The mounting bracket 1192 includes a cylindrical body 1194 and a slide plate 1196 that extends outwardly from the body 1194. As shown in FIG. 29, an aperture 1198 is defined at one end of the cylindrical body 1194, which receives the proximal end 1190 of the shaft 1136 and provides a passageway through which the connecting wire 1170 passes to connect the conductor plate 1164 to the other electrical circuitry 1148.

As shown in FIG. 28, the edges of the slide plate 1196 are received in a pair of guide slots 1200 defined in the handle 1132, which guide the movement of the mounting bracket 1192 as the needle tip 1126 is retracted. A biasing element such as, for example, a spring 1202 positioned between the slide plate 1196 and the distal handle end 1134. In the illustrative embodiment, the spring 1202 is configured to bias the slide plate 1196 away from the distal handle end 1134 and hence bias the needle tip 1126 is the retracted position.

A rod 1204 extends between the cylindrical body 1194 and the locking arm 1184. As shown in FIG. 29, the rod 1204 is received in an aperture 1206 defined in the locking arm 1184. The locking arm 1184 includes a sleeve 1208 positioned in the aperture 1206, and the rod 1204 engages the sleeve 1208 when the needle shaft 1136 is an extended position. In the illustrative embodiment, the sleeve 1208 is formed from a metallic material such as, for example, steel. A pivot pin 1212 extends outwardly from the lower housing 1142 and is received in a bore defined in the locking arm 1184 near an end 1216. The retraction mechanism 1160 also includes another biasing element, illustratively embodied as an elastic band 1220, which is coupled to the shaft end 1216 and the lower housing 1142.

When the needle shaft 1136 is in its extended position and ready for insertion into a patient's tissue, the sleeve 1208 is initially engaged with the rod 1204, as shown in FIG. 29. The band 1220 applies a force to the locking arm 1184 to bias in the position shown in FIG. 29 to keep the rod 1204 engaged with the sleeve 1208, thereby resisting the force exerted by the spring 1202 against the slide plate 1196 and maintaining the needle shaft 1136 in the extended position.

As described above, the automatic needle retraction mechanism 1130 is operable to quickly retract the needle tip 1126 a short distance after the needle tip 1126 has penetrated the tissue. To do so, the linear actuator 1180 is energized to advance its shaft 1182 into contact with the locking arm 1184, thereby causing the arm 1184 to pivot about the pin 1212 as indicated by arrow 1222. As the arm 1184 pivots, the end of the rod 1204 disengages from the sleeve 1208 and moves toward the center of the aperture 1206. When the rod 1204 disengages from the sleeve 1208, the spring 1202 urges the mounting bracket 1192 in the direction indicated by arrow 1224 in FIG. 29. As the mounting bracket 1192 moves, the needle tip 1126 retracts away from the opposite wall of the patient's lumen.

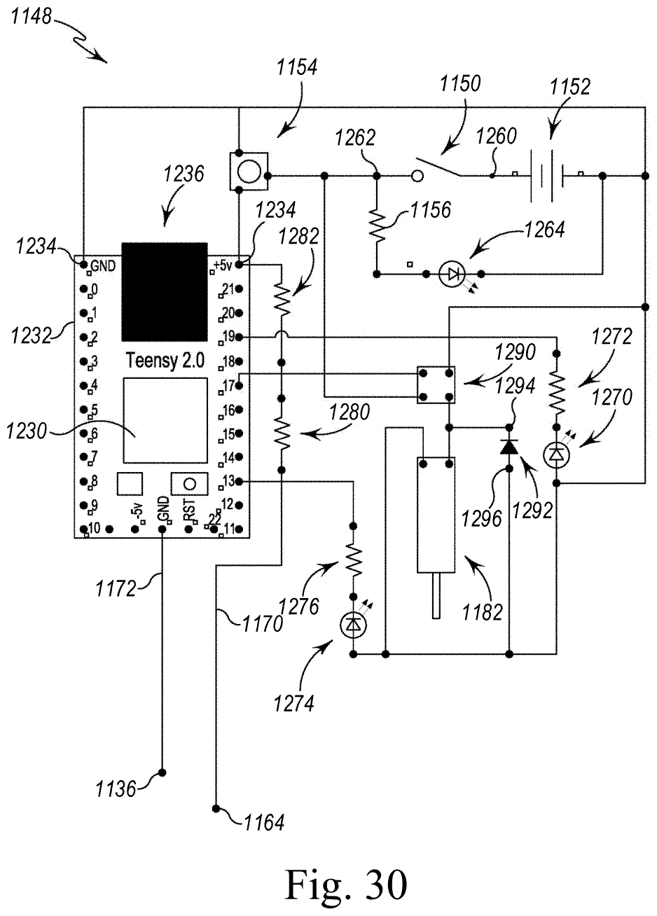

Referring now to FIG. 30, the electrical circuitry 1148 is shown. As described above, the electrical circuitry 1148 is operable to detect a change in electrical resistance that is produced when the needle tip 1126 exits one type of tissue and enters another type of tissue or lumen, as described in greater detail below. In that way, the electrical circuitry 1148 functions as a sensor.

The circuitry 1148 includes a microprocessor 1230 such as, for example, an 8-Bit AVR 16 MHz Processor (ATMEGA32U4), which is commercially available from Atmel Corporation. The microprocessor 1230 is attached a circuit 1232 that also includes various terminals 1234 connected to other circuitry 1148. An I/O port 1236 such as, for example, a USB port, is attached to the circuit 1232 to permit a user to upload software and data to, and download from, the microprocessor 1230. Illustratively, the microprocessor 1230, the circuit 1232, and the I/O port 1236 are available in a Teensy 2.0 USB-based microcontroller development system. A voltage supply includes a single 9 VDC battery 1152, the anode of which is coupled to one terminal 1260 of the power switch 1150. The other terminal 1262 of switch 1150 is coupled to a voltage regulator 1154 and to the anode of a “Power Indicator” LED 1264 of the LEDs 1146 through a 220Ω resistor 1156. As shown in FIG. 30, the cathode of the Power Indicator LED 1264 is coupled to the cathode of the battery 1152 and to the GND terminal of the circuit 1232. In the illustrative embodiment, the voltage regulator 1154 is a Texas Instruments LP2981 regulator. The voltage regulator 1154 is connected to the 5 V terminal of the circuit 1232 and is configured to condition the 9 VDC battery voltage to 5 volts.

The circuitry 1148 also includes a “Low Battery” LED 1270, which is energized by the microprocessor 1230 when battery voltage drops below a predetermined threshold. The cathode of the LED 1270 is connected through a 220Ω resistor 1272 to the “13” terminal of the circuit 1232. The anode of the LED 1270 is connected to the GND terminal of the circuit 1232 and an anode of the penetration indicator LED 1274. The cathode of the LED 1274 is connected to the “13” terminal of the circuit 1232 through another 220Ω resistor 1276. A battery monitor (not shown) may be connected to another terminal of the circuit 1232.

The shaft 1136 of the instrument 1112 is coupled via a wire 1172 to a ground terminal of the circuit 1232. The conductor plate 1164 in the tip 1126 is coupled via a wire 1170 through a 68Ω resistor 1280 and a 100 kΩ resistor 1282 to the “18” terminal and the 5 V terminal of the circuit 1232. The shaft 1136 and the plate 1164 form part of the sensor circuit used to detect when the needle tip 1126 has penetrated a lumen. It should be appreciated that in other embodiments the sensor circuit may include a pair of conductor plates, which are electrically isolated from one another, and the elongated shaft may be formed from a non-conductive material.

The linear actuator 1180 is connected to the anodes of the LEDs 1270, 1274 and the GND terminal of the circuit 1232. The linear actuator 1180 is also connected to a relay switch 1290, which is positioned between the actuator 1180 and the terminal 1262 of the switch 1150. The relay switch 1290 is also connected to the “17” terminal of the circuit 1232 and to the GND terminal, as shown in FIG. 30. The circuitry 1148 also includes a snubber diode 1292 that is connected between the positive and negative poles of the actuator 1180 and the power supply 1152. As shown in FIG. 30, the cathode 1294 of the diode 1292 is connected to the relay switch 1290, while the anode 1296 of the diode 1292 is connected to the linear actuator 1180 and the power supply 1152.

Illustratively, the microprocessor 1230 applies 4.7 VDC to the conductor plate 1164 while the shaft 1136 is connected to ground (e.g., the user's hand). The microprocessor 1230 is programmed to measure the electrical resistance in the circuit 1232 at a controlled distance. In the illustrative embodiment, the distance is equal to a 0.5 millimeter gap between the conductor plate 1164 and the cutting end of the shaft 1136 that is created the non-conductive film 1168. In the illustrative embodiment, the 0.5 millimeter gap corresponds to the thickness of the film ring 1168. During operation, when the conductor plate 1164 exits the patient's tissue and enters a liquid-filled or empty target lumen, the resistance sensed at the conductor plate 1164 experiences a “step” change, which the microprocessor 1230 is programmed to register as indicating, for example, that the tip 1126 has penetrated a lumen. The microprocessor 1230 is programmed to switch the “13” terminal continuously “high,” thereby turning the indicator LED 1274 continuously “on.”

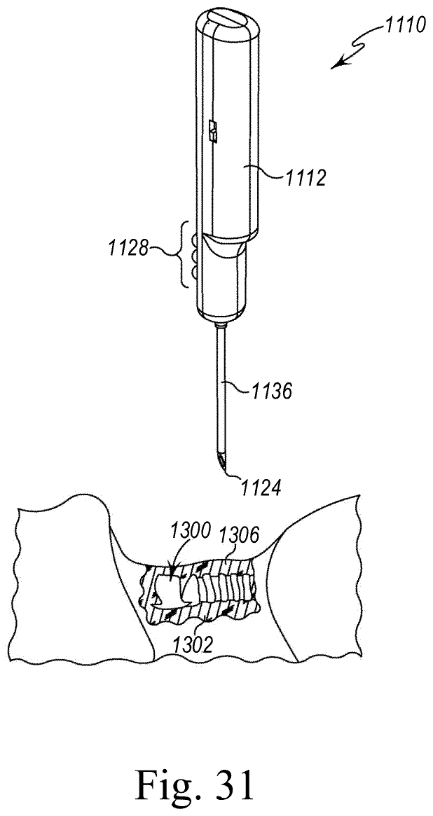

In use, the needle tip 1126 of the surgical instrument 1112 may be used to form a puncture in a patient's issue. As shown in FIG. 31, a surgeon or other user may align the needle tip 1126 with the target lumen of the patient's body (in this case, a patient's trachea 1300) and toggle the power switch 1150 to energize the sensor circuit formed by the microprocessor 1230, the conductor shaft 1164, and the outer cannula 1136. Initially, when the needle tip 1126 is out of contact with the patient's tissue, the circuit is open and the resistance value effectively infinite.

Once the needle tip 1126 is properly aligned, it may be advanced into contact with the patient's tissue and through the anterior wall 1306. When the needle tip 1126 engages the patient's tissue, the circuit is closed, and the resistance value measured by the microprocessor 1230 enters a predetermined range. In the illustrative embodiment, the range is between 1 kilo-ohm and 100 kilo-ohms. It should be appreciated that in other embodiments other ranges of resistance values may be used. The controller 1230 activates a timer when the resistance value enters the predetermined range, and after a predetermined amount of time, the microprocessor 1230 activates the LED 1274. In the illustrative embodiment, the predetermined amount of time is 200 milliseconds. When the microprocessor 1230 activates the LED 1274 in the illustrative embodiment, the microprocessor 1230 is programmed to consecutively toggle the “13” terminal “high” and “low,” thereby causing the LED 1274 to flash “on” and “off” to indicate to the user that the instrument 1112 is armed.

In other embodiments, other sensors may be used to determine when the instrument 1112 is properly positioned and ready to be armed. For example, the instrument 1112 may include a pressure sensor that measures the pressure on the needle tip such that when the pressure surpasses the amount of pressure associated with penetrating the patient's tissue, the controller would activate the indicator and arm the instrument 1112. In other embodiments, the instrument 1112 may also include a cancel switch that the user may toggle to disarm the instrument 1112.

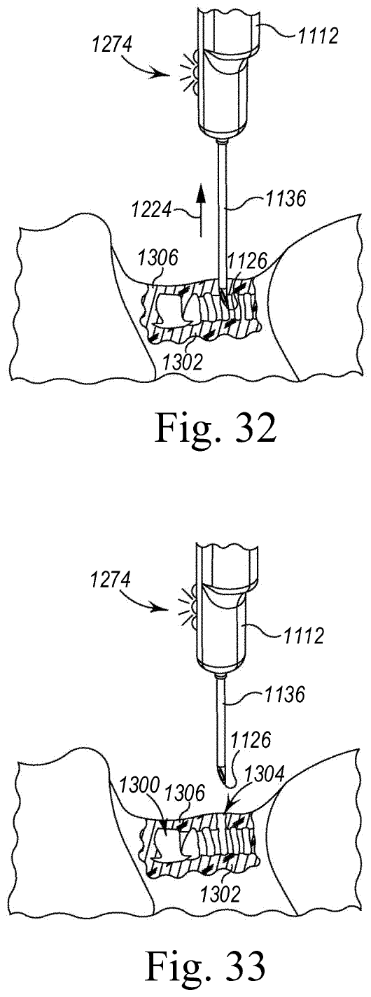

As the needle 1126 is advanced into the target lumen, the conductor plate 1164 remains engaged with the patient's tissue. When the needle 1126 reaches, and protrudes into, the target lumen (e.g., the trachea 1300, esophagus, or spinal column) as shown in FIG. 32, the resistance at the conductor plate 1164 changes sharply. In the case of a trachea, the sensor circuit effectively opens. When the resistance value passes a predetermined threshold, and the microprocessor 1230 is programmed to switch the “13” terminal continuously “high,” thereby turning the indicator LED 1274 continuously “on” to inform the user that the needle 1126 has reached the lumen. In the illustrative embodiment, the threshold is 100 kilo-ohms or greater.

The microprocessor 1230 is also programmed to switch the “17” terminal to “high” after a preset delay, thereby activating the relay switch 1290. It should be appreciated that in other embodiments the preset delay may be omitted and the switch 1290 activated immediately. When the switch 1290 is activated, it connects the linear actuator 1180 to the battery 1152, thereby energizing the actuator. As described above, the actuator 1180 is operable to advance its output shaft 1182 into contact with the locking arm 1184 and causing the locking arm 1184 to pivot. As the arm 1184 pivots, the end of the rod 1204 disengages from the sleeve 1208 and moves toward the center of the aperture 1206. When the rod 1204 disengages from the sleeve 1208, the spring 1202 urges the mounting bracket 1192 in the direction indicated by arrow 1224 in FIG. 29. As the mounting bracket 1192 moves, the needle tip 1126 retracts in direction shown in FIG. 32, away from the opposite wall 1302 of the patient's trachea 1300 and out of the incision 1304, as shown in FIG. 33.

In other embodiments, the actuator may be embodied as an electric motor, electromagnet, or other electromechanical device operable to move the locking arm 1184 within a sufficient period of time after the microprocessor detects penetration of the lumen. In the illustrative embodiment, the actuator 1180 is operable to move the locking arm 1184 such that the needle is retracted in 100 milliseconds.

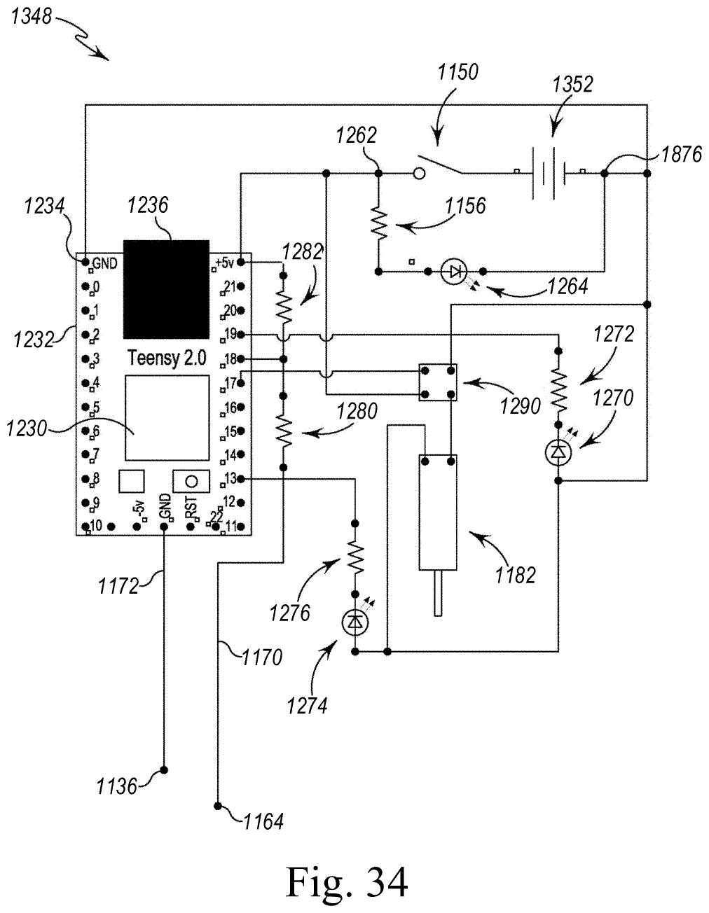

Referring now to FIG. 34, another embodiment of electrical circuitry 1348 is illustrated. The electrical circuitry 1348 is identical to the circuitry 1148 described above, except for the use of two 3 VDC batteries and the omission of a voltage regulator and snubber diode. As shown in FIG. 34, the anodes of the two 3 VDC batteries 1352 are coupled to one terminal 1260 of the power switch 1150. The other terminal 1262 of switch 1150 is coupled to the 5 V terminal of the circuit 1232 and to the anode of the “Power Indicator” LED 1264 of the LEDs 1146.

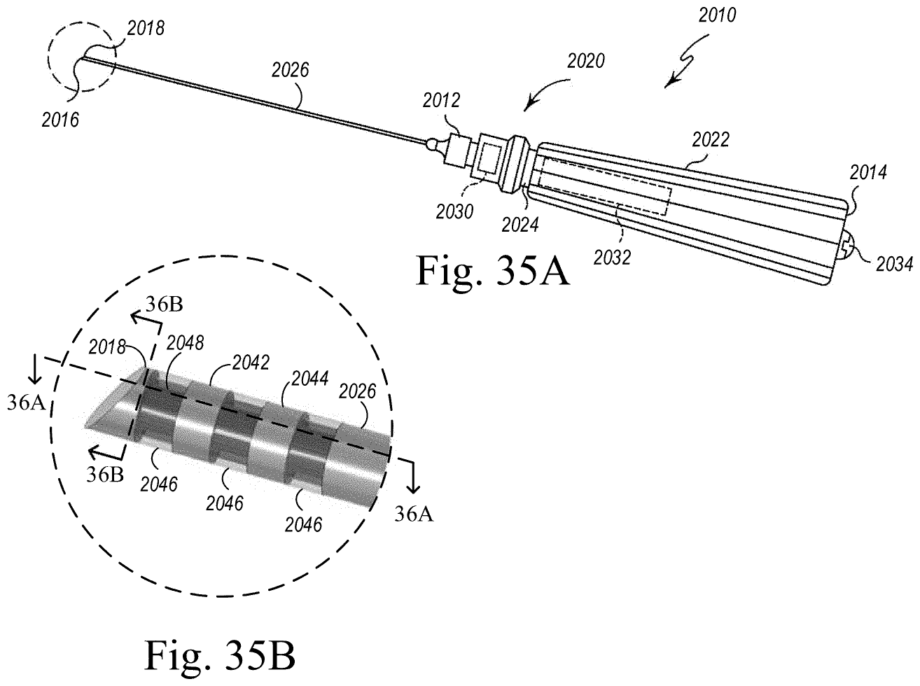

Referring now to FIGS. 35-38, another surgical instrument system including a puncture instrument 2010 configured for insertion into the soft tissue of a patient is illustrated. The surgical instrument 2010 includes an elongated body 2012 that extends from a proximal end 2014 to a distal end 2016. A needle tip 2018 configured to pierce the tissue is formed at the distal end 2016 of the body 2012. The surgical instrument 2010 includes an indicator 2020 configured to notify a user that the needle tip 2018 has penetrated the tissue. In the illustrative embodiment, the surgical instrument 2010 detects increases and decreases in capacitance and can determine what tissue the needle tip 2018 is in. The surgical instrument may activate the indicator 2020 to indicate which tissue the needle tip 2018 is in.

The elongated body 2012 includes a handle 2022 extending from the proximal end 2014 to a distal handle end 2024. A shaft 2026 extends from the handle 2022 to the needle tip 2018. In the illustrative embodiment, the shaft 2026 is a cannula formed from a non-conductive material such as, for example, a non-conductive ceramic or plastic material. The needle tip 2018 and the shaft 2026 are integrally formed, but it should be appreciated that in other embodiments the needle tip 2018 and the shaft 2026 may be formed as separate components that are assembled.

As illustrated in FIG. 35A, the indicator 2020 is positioned at the distal handle end 2024. In the illustrative embodiment, the indicator 2020 includes a light source such as, for example, an LED 2030, and other electrical circuitry 2032 operable to energize the LED 2030 to provide a visual output to the user. In other embodiments, the indicator 2020 may include other electrical circuitry to provide an audible output to the user. The surgical instrument 2010 also includes a power switch 2034 positioned at the proximal end 2014 of the handle 2022. Switch 2034 is operable to supply power to the electrical circuitry 2032 including LED 2030.

The electrical circuitry 2032 may be embodied as any suitable circuitry configured to perform the function described herein. The electrical circuitry 2032 may include processors, memory, microcontrollers, wires, etc. In some embodiments, the electrical circuitry 2032 may include wired or wireless communication components, allowing for communication with other electronic devices, such as a computer, cell phone, etc. In such embodiments, some of the function described herein may be performed on the remote electronic device, such as performing calculations and determinations in regard to what tissue the needle tip 2018 is in. In some cases, the electrical circuitry 2032 may include one or more components that are outside of the surgical instrument 2010. In such embodiments, the various components of the electrical circuitry may be connected by a wired or wireless connection.

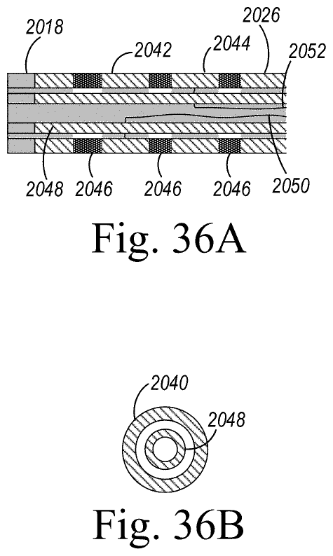

As illustrated in FIG. 35B, the electrical circuitry 2032 also includes one or more capacitive sensors, such as a lead capacitive sensor 2042 and reference capacitive sensor 2044. In the illustrative embodiment, each capacitive sensor 2042, 2044 may be embodied as a ring of conductive material, such as steel or aluminum. In the illustrative embodiment, each capacitive sensor 2042, 2044 may have an outer and inner diameter similar to that of the shaft 2026, such as an inner diameter of 0.1 to 3 millimeters and an outer diameter of 0.3 to 4.5 millimeters, with a wall thickness of 0.05 to 0.4 millimeters. The width of each capacitive sensor 2042, 2044 may be any suitable value, such as 0.1-5 millimeters. In some embodiments, those dimensions of the capacitive sensors 2042, 2044 may be larger or smaller. In the illustrative embodiments, the lead capacitive sensor 2042 is positioned close to the needle tip 2018, such as within 0.1-1 millimeter away from the needle tip 2018. The reference capacitive sensor 2044 may be placed relatively close to the lead capacitive sensor 2042, such as with a 1-5 millimeters gap between them. In the illustrative embodiment, both the lead capacitive sensor 2042 and the reference capacitive sensor 2044 are positioned within 5-25 millimeters of the needle tip 2018. Each capacitive sensor 2042, 2044 may be connected to the rest of the circuitry 2032 in the handle 2022 by an electrical connector 2050, 2052, as shown in FIG. 36A. It should be appreciated that, when a patient's tissue contacts the capacitive sensors 2042, 2044, the electrical circuitry 232 is operable to detect the electrical capacitance of the tissue surrounding the capacitive sensors 2042, 2044, as described in greater detail below. It should further be appreciated that capacitive sensor 2042 may be in different tissue from capacitive sensor 2044, and the electrical circuitry 232 may in some embodiments be operable to determine whether the lead capacitive sensor 2042 is in different tissue (or lumen) than the reference capacitive sensor 2044.

In the illustrative embodiment, each capacitive sensor 2042, 2044 is isolated from each other and from the shaft 2026 and needle tip 2018 by nonconductive spacers 2046. The nonconductive spacers 2046 may be formed from any suitable material, such as plastic, ceramic, or glass. In some embodiments, the needle tip 2018 may be a capacitive sensor as well. In the illustrative embodiment, there is a central tube 2048 disposed in the shaft 2026. The central tube 2048 may be conductive and may be connected to a ground voltage level of the surgical instrument 2010. The central tube 2048 may act as a ground guard shield for each capacitive sensor 2042, 2044.

As illustrated in FIG. 36A, the electrical connectors 2050, 2052 may be connected to the capacitive sensors 2042, 2044. In the illustrative embodiment, the electrical connectors 2050, 2052 may be wires, such as copper wires. Additionally or alternatively, the electrical connectors 2050, 2052 may be strips, tubes, or any other suitable electrical connector. As shown in FIG. 36A, in the illustrative embodiment, the electrical connectors 2050, 2052 may pass through an opening in the central tube 2048 (not shown) and may extend along the central tube 2048 toward, e.g., the circuitry 2032 in the handle 2022. In the illustrative embodiment, each of the electrical connectors 2050, 2052 is insulated, such as with a plastic sleeve, in order to prevent the electrical connectors 2050, 2052 from touching another conductive object, such as each other, the central tube 2048, etc.

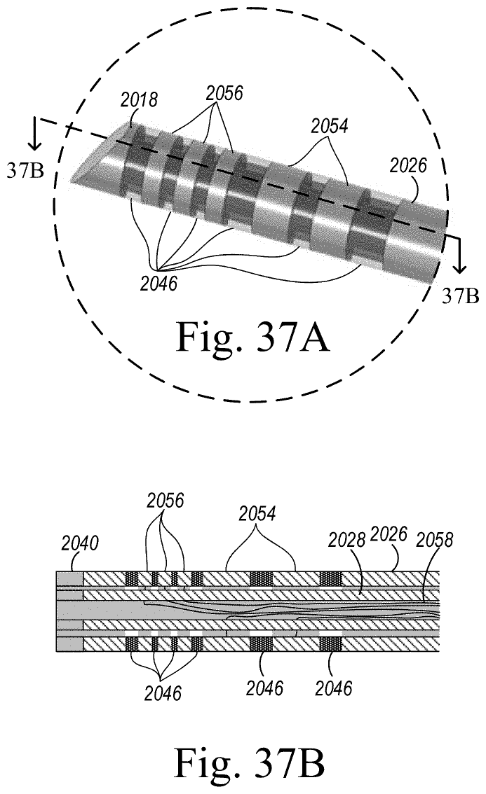

In some embodiments, as illustrated in FIGS. 37A and B, the electrical circuitry 2032 may include several capacitive sensors of different sizes, such as high capacitance sensors 2054 and low capacitance sensors 2056. The low capacitance sensors 2056 may be more sensitive to small changes in position or in capacitance but may be more susceptible to noise. Conversely, the high capacitance sensors 2054 may be less sensitive to small changes but may be less sensitive to noise. By combining both in one embodiment, the electrical circuitry 2032 may sense small changes in position and in capacitance while being less susceptible to noise. Each high capacitance sensor 2054 and low capacitance sensor 2056 may be connected to an electrical connector 2058. Each electrical connector 2058 may be similar to the electrical connectors 2050, 2052.

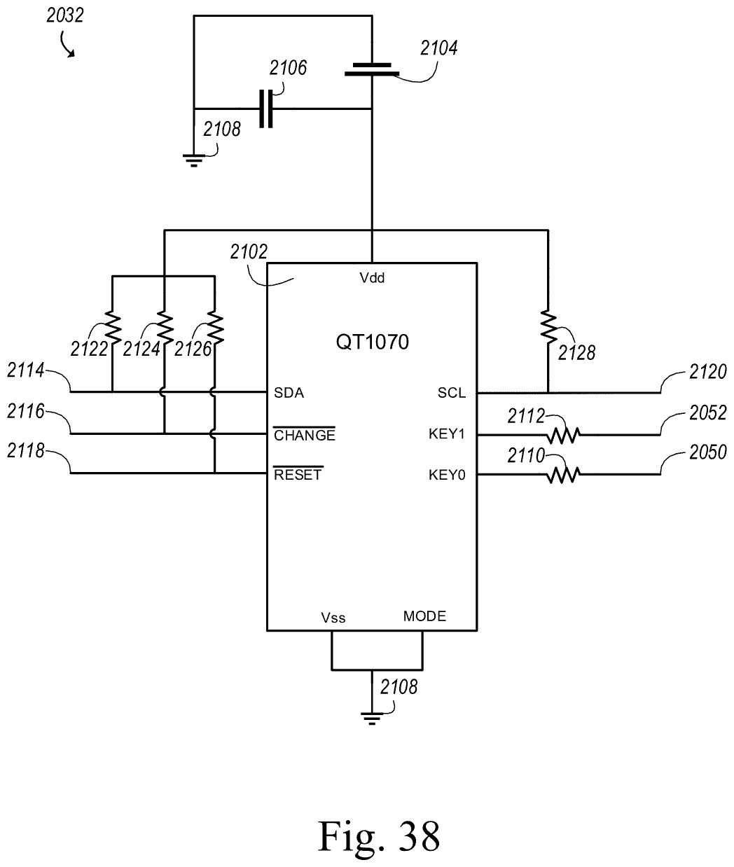

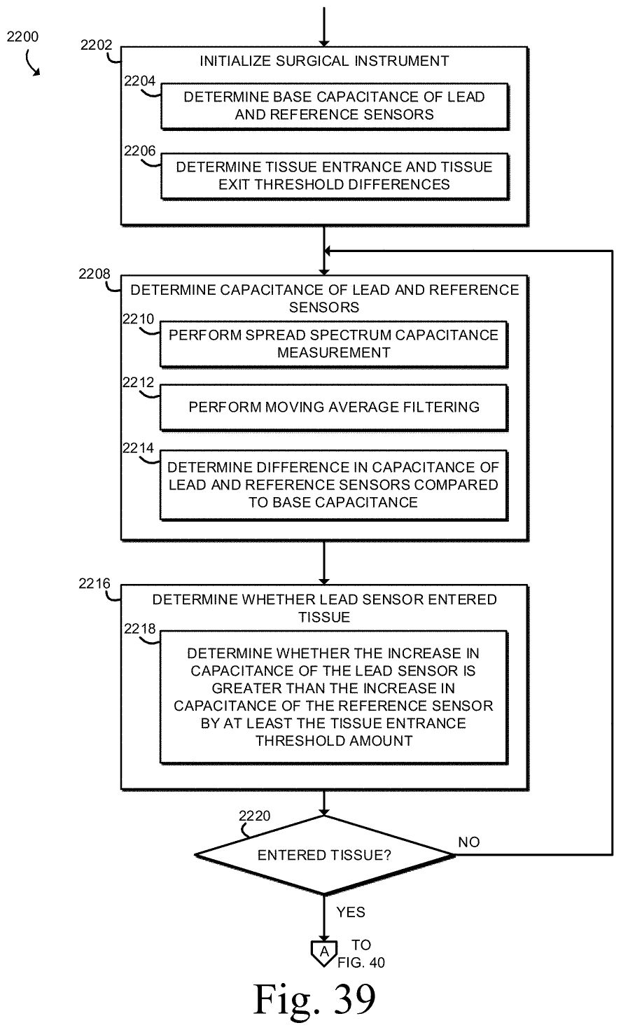

Referring now to FIG. 38, the electrical circuitry 2032 is shown in greater detail. The schematic and block circuit diagram descriptions that follow identify specific integrated circuits and other components of the circuitry 2032 and in many cases specific sources for these. Specific terminal and pin names and numbers are generally given in connection with these for the purposes of completeness. It is to be understood that these terminal and pin identifiers are provided for these specifically identified components. It is to be understood that this does not constitute a representation, nor should any such representation be inferred, that the specific components, component values or sources are the only components available from the same or any other sources capable of performing the necessary functions. It is further to be understood that other suitable components available from the same or different sources may not use the same terminal/pin identifiers as those provided in this description. In some instances, specific values or ranges of values may be specified for voltage levels, resistor values, capacitor values, etc. Those values may be used in the illustrative embodiment, but, in other embodiments, different values may be used for those components.

The circuitry 2032 includes a capacitance monitoring circuit 2102, for example, an AT42QT1070 seven-channel QTouch® touch sensor integrated circuit available from Atmel. A voltage supply includes a battery 2104 with a voltage anywhere from 1.8-5.5 volts and an optional capacitor 2106 with about a 0.1 microfarad capacitance connected in parallel. The anode of the battery 2104 is coupled to a ground 2108, which may be a chassis ground, signal ground, or earth ground. The cathode of the battery 2104 is coupled to the voltage input of the capacitance monitoring circuit 2102. Electrical connector 2050 may be connected to key 0 input in series with resistor 2110, and electrical connector 2052 may be connected to key 1 input in series with resistor 2112. Each resistor 2110, 2112 may have a resistance from 4.7 kΩ to 20 kΩ.

The capacitance monitoring circuit 2102 has additional inputs connected to wires 2114, 2116, 2118, and 2120. Wire 2114 is connected to a serial data port (SDA). Wire 2116 is connected to a change port indicative of a change in status of an input. Wire 2118 is connected to a reset port. Wire 2120 is connected to a serial data clock (SCL). Wires 2114, 2116, 2118, and 2120 are connected in series to resistors 2122, 2124, 2126, and 2128, respectively, and then to the cathode of the battery 2104. The SCL port acts as a clock for serial communication on the SDA port. Communication with the SCL and SDA port may be performed in accordance with the I²C (inter-integrated circuit) protocol. Each of the resistors 2122, 2128 may be selected to comply with the timing requirements of I²C, such as 1-10 kΩ. Each of resistors 2124 and 2126 may be any suitable value, such as 47 kΩ. The current sink port Vₛₛ and the mode selection port are connected to ground 2108.

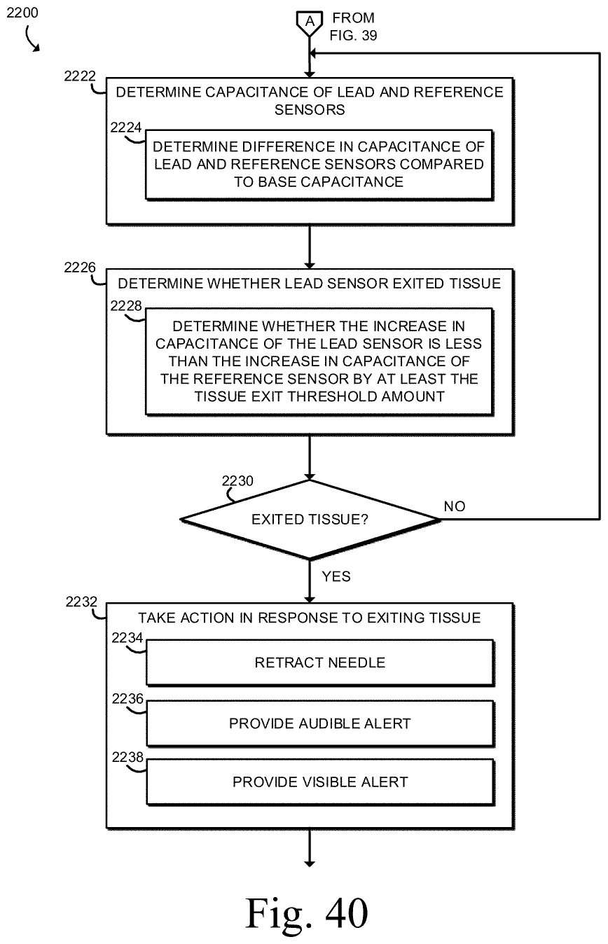

Referring now to FIG. 39, in use, electrical circuitry 2032 may perform a method 2200 for sensing a lumen of a patient. The method 2200 begins in block 2202, in which the electrical circuitry 2032 initializes the surgical instrument 2010. The electrical circuitry 2032 may determine a base capacitance of a lead capacitive sensor 2042 and a reference capacitive sensor 2044 in block 2204, such as by measuring the capacitance or accessing stored base capacitance values. As used herein, a base capacitance of capacitive sensors refers to the capacitance value when the capacitive sensor is not near a patient's tissue or other material that may affect the measured capacitance difference of the capacitive sensor. For example, the base capacitance of capacitive sensors 2042, 2044 may be measured when the shaft 2026 and needle tip 2018 are in air. The electrical circuitry 2032 may determine a tissue entrance threshold difference and a tissue exit threshold difference, which are described in more detail below. The thresholds may be determined by accessing information stored in the electrical circuitry, by performing a measurement, or in any other suitable manner.

In block 2208, the electrical circuitry 2032 determines the capacitance of the lead capacitive sensor 2042 and reference capacitive sensor 2044. In some embodiments, the electrical circuitry 2032 may perform a spread spectrum capacitance measurement in block 2210. The spread spectrum measurement measures the capacitance at several different frequencies, which may provide additional information in regard to what tissue is surrounding the capacitive sensor and may also reduce noise. In block 2212, the electrical circuitry 2032 may perform moving average filtering, such as by averaging out the previous ten measurements. In block 2214, the electrical circuitry 2032 may determine a difference in capacitance of each of the capacitive sensors 2042, 2044 relative to the base capacitance. For example, the base capacitance when surrounded by air of the lead capacitive sensor 2042 may be 15 picofarad, and, when surrounded by skin tissue, the capacitance of the lead capacitive sensor 2042 may be 20 picofarad, leading to a calculated increase in capacitance of 5 picofarad. It should be appreciated that, in some cases, the measured capacitance value of each of the lead capacitive sensor 2042 and the reference capacitive sensor 2044 may change in a correlated manner due to external factors such as electrical interference, but a change in measured capacitance value of, e.g., the lead capacitive sensor 2042 without a corresponding change in measured capacitance value of the reference capacitive sensor 2044 is more likely to indicate a change in the local conditions of the lead capacitive sensor 2042 relative to the reference capacitive sensor 2044, such as by being in a different surrounding material such as tissue.

In block 2216, the electrical circuitry 2032 may determine whether the lead capacitive sensor 2042 has entered tissue of a patient. In the illustrative embodiment, the electrical circuitry 2032 may determine whether an increase in the capacitance value of the lead capacitive sensor 2042 over the base capacitance value of the lead capacitive sensor 2042 is greater by at least the tissue entrance threshold compared to the increase in the capacitance value of the reference capacitive sensor 2044 over the base capacitance value of the reference capacitive sensor 2044 in block 2218.

For example, the tissue entrance threshold may be 5 picofarad, the base capacitance of the lead capacitive sensor 2042 may be 15 picofarad, and the base capacitance of the reference capacitive sensor 2044 may be 18 picofarad. In operation, but before any part of the needle tip 18 has entered the patient's tissue, the measured capacitance of the lead capacitive sensor 2042 and reference capacitive sensor 2044 may be 20 picofarad and 22 picofarad, respectively, with an increase of 4 or 5 picofarad due to correlated fluctuations from interference. The increase of the capacitance of the lead capacitive sensor 2042 (of 5 picofarad) relative to the increase of the capacitance of the reference capacitive sensor (of 4 picofarad) would then be 1 picofarad, below the tissue entrance threshold of 5 picofarad, indicating that the lead capacitive sensor 2042 has not entered the patient's tissue. Once the needle tip 18 and the lead capacitive sensor enters the tissue of the patient, the measured capacitance of the lead capacitive sensor 2042 may increase to 30 picofarad, and the measured capacitance of the reference capacitive sensor 2044 may stay at 22 picofarad. The increase of the capacitance of the lead capacitive sensor 2042 (of 15 picofarad) relative to the increase of the capacitance of the reference capacitive sensor (of 4 picofarad) would then be 11 picofarad, above the tissue entrance threshold of 5 picofarad, indicating that the lead capacitive sensor 2042 has entered the tissue of the patient.

In block 2220, if the electrical circuitry 2032 determines that the lead capacitive sensor 2042 has not entered the patient's tissue, the method 2200 will loop back to block 2208 to continue monitoring the capacitive sensors. If the electrical circuitry 2032 determines that the lead capacitive sensor 2042 has entered the patient's tissue, the method 2200 proceeds to block 2222 in FIG. 40.

Referring now to FIG. 40, in block 2222, the electrical circuitry 2032 again determines the capacitance of the lead capacitive sensor 2042 and the reference capacitive sensor 2044. As in block 2208, the electrical circuitry 2032 may employ spread spectrum capacitive measurement and/or may perform moving average filtering. As in block 2214, the electrical circuitry 2032 may determine a difference in capacitance of each of the capacitive sensors 2042, 2044 relative to the base capacitance in block 2224.

In block 2226, the electrical circuitry 2032 determines whether the lead capacitive sensor 2042 has exited the patient's tissue, such as into a lumen. Similar to block 2218, in the illustrative embodiment, the electrical circuitry 2032 may determine whether a decrease in the capacitance value of the lead capacitive sensor 2042 under the base capacitance value of the lead capacitive sensor 2042 is greater by at least the tissue exit threshold compared to a decrease in the capacitance value of the reference capacitive sensor 2044 under the base capacitance value of the reference capacitive sensor 2044 in block 2228.

In block 2230, if the electrical circuitry 2032 determines that the lead capacitive sensor 2042 has not exited the patient's tissue, the method 2200 loops back to block 2222 to continue monitoring of the capacitance of the lead capacitive sensor 2042 and the reference capacitive sensor 2044. If the electrical circuitry 2032 determines that the lead capacitive sensor 2042 has exited the patient's tissue, the method 2200 proceeds to block 2232, in which the electrical circuitry 2032 takes same action in response to exiting the patient's tissue. For example, in the illustrative embodiment, the electrical circuitry may active an actuator to retract the shaft 2026 in block 2234. The actuator may retract the shaft 2026 any suitable amount, such a 1 to 10 millimeters. Additionally or alternatively, in some embodiments, the electrical circuitry 2032 may provide an audible alert in block 2236 and/or may provide a visible alert in block 2238. Of course, in some embodiments, additional or alternative actions may be taken, such as sending a notification to a remote device or controlling some other action on the surgical instrument 2010.

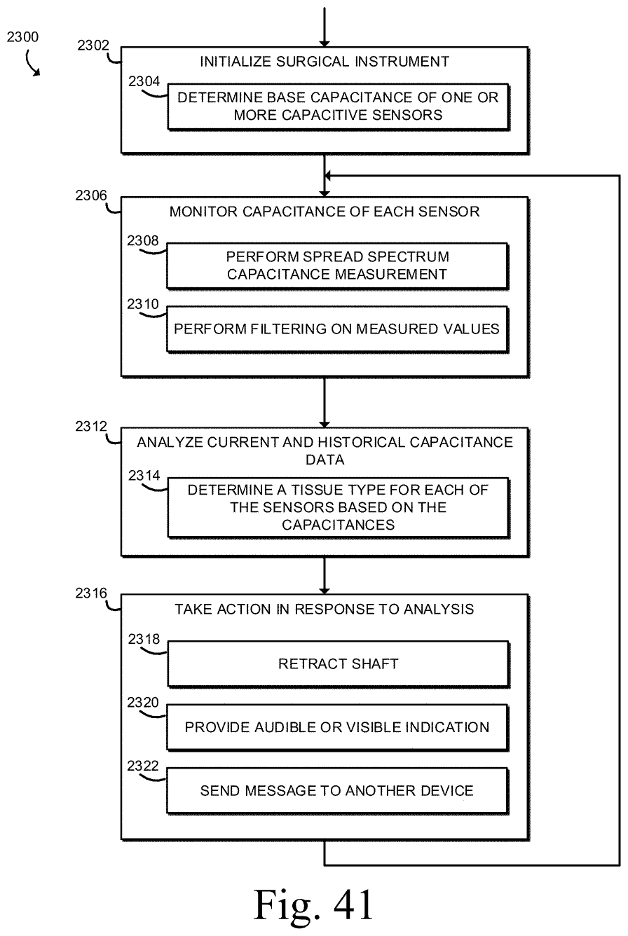

Referring now to FIG. 41, in use, electrical circuitry 2032 may perform a method 2300 for analyzing capacitive sensor data of the surgical instrument 2010. The method 2300 begins in block 2302, in which the electrical circuitry 2032 initializes the surgical instrument 2010. The electrical circuitry 2032 may determine a base capacitance of one or more capacitive sensors in block 2304, such as by measuring the capacitance or accessing stored base capacitance values.

In block 2306, the electrical circuitry 2032 monitors the capacitance of each capacitive sensor. In some embodiments, the electrical circuitry 2032 may perform a spread spectrum capacitance measurement in block 2308. In block 2310, the electrical circuitry 2032 may perform moving average filtering, such as by averaging out the previous ten measurements.

In block 2312, the electrical circuitry 2032 analyzes the current and historical capacitance data of the capacitive sensors. For example, the electrical circuitry 2032 may determine absolute changes in capacitance for each of the capacitive sensors, determine relative differences in capacitance or changes in capacitance of one capacitive sensor compared to others, determine a rate of change of capacitance, determine a pattern present in the capacitance of different capacitive sensors at different locations and/or times, etc. In the illustrative embodiment, the electrical circuitry 2032 may determine a tissue type of one or more of the capacitive sensors based on the measured capacitance values in block 2314. It should be appreciated that different capacitive sensors may be in different tissues at the same time, and the electrical circuitry 2032 may be able to determine which parts of the shaft 2026 are in what tissue. It should further be appreciated that the electrical circuitry 2032 need not be limited to sensing only whether a capacitive sensor is in tissue or air. Rather, the electrical circuitry 2032 may be able to determine which type of tissue a capacitive sensor is in, such as skin, fat, blood, muscle, lung, etc. The ability to determine a location of various parts of the shaft 2026 including the needle tip 2018 combined with the ability to control the position of the needle tip 2018 (i.e., by retracting it a controllable amount) will offer a unique, and, more importantly, safe entry into the body during tracheostomy, establishment of safe pneumoperitoneum in laparoscopic surgery, gastrostomy feeding tube placement, and epidurals.

In block 2232, the electrical circuitry 2032 takes same action in response to the analysis. For example, in the illustrative embodiment, the electrical circuitry may active an actuator to retract the shaft 2026 in block 2318. Additionally or alternatively, in some embodiments, the electrical circuitry 2032 may provide an audible or visible alert in block 2320. In some embodiments, the electrical circuitry may send a message to a remote device indicative of the analysis. Of course, depending on the analysis, such as when there is no change, the electrical circuitry 2032 may take no action at all. The method 2300 then loops back to block 2306 to continue monitoring the capacitance of the capacitive sensors.

It should be appreciated that although the concept of detecting a lumen in a patient's body has been described above in reference to surgical instruments that may be used to create punctures in a patient's tissue, the techniques and concepts described above may be incorporated into other surgical instruments such that entry into a lumen or movement between various tissue types may be detected. For example, any surgical cutting tool such as, for example, a cutting blade, reamer, drill, or other instrument may include circuitry to detect fluctuating levels of electrical capacitance and thereby determine when a distal end of the cutting tool has entered a lumen. Other surgical instruments such as, for example, guides, trials, probes, and so forth may also include circuitry to detect fluctuating levels of electrical capacitance and thereby determine when a distal end of the surgical instrument has entered a lumen.

While the disclosure has been illustrated and described in detail in the drawings and foregoing description, such illustration and description is to be considered as exemplary and not restrictive in character, it being understood that only illustrative embodiments have been illustrated and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

What is claimed is:

1. A surgical instrument system comprising:
a housing including a handle;
a shaft extending outwardly from the housing to a distal end configured to form a puncture in a patient's tissue, the shaft comprising a capacitive sensor;
a controller positioned in the housing, the controller configured to measure a capacitance value of the capacitive sensor.

2. The surgical instrument system of claim 1, further comprising a retraction mechanism operable to move the distal end of the shaft in a first direction toward the housing,
wherein the controller is configured to (i) determine, based on the capacitance value of the capacitive sensor, whether the shaft should be retracted, and (ii) energize the retraction mechanism to move to the distal end of the shaft in the direction toward the housing in response to a determination that the shaft should be retracted.

3. The surgical instrument system of claim 1, further comprising a light source or an audio source positioned in the housing,
wherein the controller is configured to (i) determine, based on the capacitance value of the capacitive sensor, whether an alert should be provided, and (ii) energize the light source or the audio source to provide the alert in response to a determination that the alert should be provided.

4. The surgical instrument system of claim 1, wherein the shaft further comprises a second capacitive sensor electrically isolated from the capacitive sensor,
wherein the controller is configured to measure a capacitance value of the second capacitive sensor.

5. The surgical instrument system of claim 4, wherein a central tube is positioned within the shaft.

6. The surgical instrument system of claim 5, wherein electrical wiring positioned within the central tube provides (i) an electrical connection between the controller and the capacitive sensor and (ii) an electrical connection between the controller and the second capacitive sensor.

7. The surgical instrument system of claim 5, wherein the central tube is configured to act as an electrical ground-guard-shield for the capacitive sensor the second capacitive sensor.

8. The surgical instrument system of claim 4, wherein the controller is configured to:
determine when the distal end of the shaft has engaged a first tissue layer of a patient in response to the difference between the monitored capacitance values of the capacitive sensor and the second capacitive sensor being greater than a first predetermined threshold, and
determine when the distal end has engaged a second tissue layer of a patient different from the first in response to the difference between the monitored capacitance values of the capacitive sensor and the second capacitive sensor being greater than a second predetermined threshold.

9. The surgical instrument system of claim 4, wherein the shaft further comprises a third capacitive sensor electrically isolated from the capacitive sensor and the second capacitive sensor,
wherein the third capacitive sensor has a base capacitance value at least twice a base capacitance value of the capacitive sensor.

10. A surgical instrument comprising:
a hollow shaft having a proximal end configured to couple to a housing and a distal end configured to form a puncture in a patient's tissue, wherein the hollow shaft comprises a capacitive sensor, and
an electrical connector electrically coupled to the capacitive sensor and extending along the hollow shaft from the capacitive sensor towards the proximal end of the hollow shaft.

11. The surgical instrument of claim 10, wherein the hollow shaft further comprises a second capacitive sensor, wherein the second capacitive sensor is electrically isolated from the capacitive sensor, and wherein the surgical instrument further comprises a second electrical connector electrically coupled to the second capacitive sensor and extending along the hollow shaft from the second capacitive sensor towards the proximal end of the hollow shaft.

12. The surgical instrument of claim 11, wherein each of the capacitive sensor and the second capacitive sensor are positioned within 25 millimeters of the distal end of the hollow shaft.

13. The surgical instrument of claim 11, wherein each of the capacitive sensor and the second capacitive sensor are positioned within 5 millimeters of the distal end of the hollow shaft.

14. The surgical instrument of claim 11, wherein the hollow shaft further comprises a third capacitive sensor electrically isolated from the capacitive sensor and from the second capacitive sensor, wherein the third capacitive sensor has a base capacitance value at least twice a base capacitance value of the capacitive sensor.

15. The surgical instrument of claim 11, wherein a central tube is positioned within the hollow shaft.

16. The surgical instrument of claim 11, further comprising:
the housing, wherein the proximal end of the hollow shaft is coupled to the housing; and
a controller positioned in the housing, the controller being configured to measure a capacitance value of the capacitive sensor and a capacitance value of the second capacitive sensor.

17. A method of using a surgical instrument, the method comprising:
monitoring, by electrical circuitry of the surgical instrument, a capacitance of one or more capacitive sensors disposed on a shaft of the surgical instrument;
analyzing, by the electrical circuitry, the capacitance of the one or more capacitive sensors;
determining, by the electrical circuitry and based on the analysis of the capacitance of the one or more capacitive sensors, that the shaft of the surgical instrument should be retracted; and
controlling, by the electrical circuitry, an actuator of the surgical instrument to retract the shaft.

18. The method of claim 17, wherein monitoring the capacitance of the one or more capacitive sensors comprises monitoring a capacitance of a first capacitive sensor and a capacitance of a second capacitive sensor, and wherein analyzing the capacitance of the one or more capacitive sensors comprises comparing the capacitance of the first capacitive sensor to the capacitance of the second capacitive sensor.

19. The method of claim 17, further comprising:
determining, by the electrical circuitry and based on the capacitance value of the one or more capacitive sensors, whether an alert should be provided; and
energizing, by the electrical circuitry a light source or audio source of the surgical instrument to provide the alert in response to a determination that the alert should be provided.

20. The method of claim 17, wherein each of the one or more capacitive sensors are positioned within 25 millimeters of a distal end of the shaft opposite a proximal end of the shaft that is coupled to a handle of the surgical instrument.
